(19) United States
(12) Patent Application Publication
(10) Pub. No.: US 2026/0368641 A1
(43) Pub. Date: Nov. 19, 2026

(54) SYSTEMS, METHODS, AND COMPUTER-ACCESSIBLE MEDIUM FOR IMAGE GENERATION IN SCANNERS WITH MEMORY, OR INVERSE-PROBLEM SOLUTION USING MULTI-FACETED CONTEXTUAL INFORMATION

(71) Applicant: New York University, New York, NY (US)

(72) Inventors: AHMET ARDA ATALIK, New York, NY (US); SUMIT CHOPRA, New York, NY (US); DANIEL KEVIN SODICKSON, Larchmont, NY (US)

(21) Appl. No.: 19/670,467

(22) Filed: May 7, 2026

Related U.S. Application Data
(60) Provisional application No. 63/802,887, filed on May 9, 2025.

(51) Int. Cl. G06N 20/00; G16H 30/20

(52) U.S. Cl. CPC G06N 20/00; G16H 30/20

(57) ABSTRACT

Exemplary systems, methods, and computer-accessible medium according to the exemplary embodiments of the present disclosure are provided for determining information pertaining to a structure based on measured data associated with the structure. Thus, the exemplary systems, methods, and computer-accessible medium can measure data associated with the structure, transform the measured data into the information using individualized side information, and apply a trust guidance mechanism to resolve ambiguities in the transformed information so as to prevent hallucination or overwriting of the measured data when incorporating the individualized side information.

### CROSS-REFERENCE TO RELATED APPLICATION(S)

This application relates to and claims priority from U.S. Patent Application No. 63/802,887, filed on May 9, 2025, the entire disclosure of which is incorporated herein by reference.

### STATEMENT REGARDING FEDERALLY FUNDED RESEARCH

This invention was made with government support under NIH P41 EB017183 awarded by the National Institute of Biomedical Imaging and Bioengineering. The government has certain rights in the invention.

### FIELD OF THE DISCLOSURE

The present disclosure relates to the field of medical imaging, and more specifically, to systems, methods, and computer accessible media for reconstructing undersampled images with side information, including side information in the form of prior scans of the same subject from prior imaging sessions, images of different contrast from the same imaging session, images from different imaging modalities, and non-image contextual data. More broadly, it relates to the solution of inverse problems using side information.

### BACKGROUND INFORMATION

Magnetic Resonance Imaging (MRI) is a mainstay of medical diagnostic imaging, due to its flexibility, its rich information content, and its excellent soft-tissue contrast. An MR scanner collects measurements in frequency space (e.g., k-space) that encode the body's response to applied electromagnetic fields, typically with multiple receiver coils capturing distinct views modulated by their individual sensitivities, and this process is mathematically described by a linear map called the forward operator. The acquired k-space measurements are then used to reconstruct a spatially resolved image by solving the corresponding linear inverse problem (LIP). Despite MRI's superior diagnostic capabilities, this modality and technology can be comparatively time-consuming and costly, which limits its overall accessibility. Reducing the time it takes to acquire an MR scan is an important practical problem that can improve patient care by limiting patient discomfort, reducing costs, and improving accessibility of this imaging modality. One exemplary way to reduce scan time is to acquire fewer k-space measurements. The challenge then becomes reconstructing high-quality images from limited data by solving the corresponding ill-conditioned/ill-posed LIP, which admits many mathematically feasible solutions, most of which fail to capture essential anatomical and clinical details accurately (see FIG. 1A).

Researchers have suggested various solutions, including compressed-sensing-based methods (see, e.g., Ref. 2) and priors learned from exemplary data or directly from the measurements themselves (see, e.g., Refs. 3 and 4). Recent advances in machine learning, and particularly deep learning (DL), have markedly improved the ability to tackle ill-posed or ill-conditioned problems. Notable early examples include the Variational Network (VarNet) approach (see, e.g., Refs. 5 and 6), the Model-Based Deep Learning (MoDL) approach (see, e.g., Ref. 7), and the FISTA-Net approach (see, e.g., Ref. 8), all of which integrate traditional optimization techniques with deep neural networks to achieve robust and efficient solutions in high-dimensional spaces. More recently, researchers have proposed generative models for reconstructing high-quality images from incomplete data (see, e.g., Refs. 9-11), and a rapidly expanding portfolio of deep-learning-based image reconstruction methods is currently under development. In all of these cases, performance at high acceleration levels is limited by the quantity of useful information that can reliably be derived about general distributions of desirable solutions. The extent to which such general information can correctly disambiguate particular solutions is also limited. Previous work has demonstrated a sharp decline in image quality at high levels of acceleration. (See, e.g., Ref. 12).

Another exemplary approach to eliminating degenerate solutions to ill-posed or ill-conditioned LIPs involves leveraging additional contextual information (e.g., relevant side information). While side information may also be incorporated via regularizers or constraints in the objective function of an optimization problem (see, e.g., Refs. 13-18), it differs from population-based regularization in that it can be specific to the particular solution of interest. The nature of such side information is problem-dependent, and in many real-world scenarios it is readily available. Relevant side information can take multiple forms, including images, text, or other types of structured data. In MR image reconstruction, for instance, the side information could be data associated with prior scans of the same patient. It could also be data gathered during the same scan, such as images obtained using an imaging pulse sequence with a different underlying contrast from the target pulse sequence. In more general settings, the side information need not be derived from the same imaging modality, nor does it need to be image-based; it could be textual (e.g., clinical notes and medical history), or even encoded features or representations learned from other related tasks or from foundation models.

An important source of individualized side information arises in longitudinal imaging studies, where a subject's prior scan, obtained in a different imaging session (optionally on a different scanner), can provide highly informative subject-specific structural context. If utilized appropriately, such longitudinal priors can disambiguate highly undersampled reconstructions beyond what population-level priors alone can offer. However, practical use is nontrivial: longitudinal scans may differ due to (a) genuine temporal changes, including pathology progression or treatment effects, (b) misalignment due to inter-scan coordinate-frame mismatch (e.g., different slice prescription and patient positioning), and (c) protocol drift or scanner differences that alter intensity statistics and contrast. These factors make naive similarity penalties or direct reference substitution unreliable and raise concerns of diagnostic accuracy, because over-reliance on the prior can suppress true interval changes or introduce plausible-looking but incorrect structures. A reconstruction framework that can leverage prior scans while constraining their influence to remain consistent with the current measurements, and that can tolerate inter-scan misalignment and protocol drift without requiring explicit pre-registration, would have wide-ranging applications.

Accordingly, there is a need to address and/or improve at least the above-described deficiencies which exist in the previous systems, methods and computer-accessible medium by providing systems, methods and computer-accessible medium for an end-to-end trainable deep learning approach that efficiently and reliably integrates side information to solve LIPs.

### SUMMARY OF EXEMPLARY EMBODIMENTS

The following is intended to be a brief summary of the exemplary embodiments of the present disclosure, and is not intended to limit the scope of the exemplary embodiments.

In some exemplary embodiments of the present disclosure, exemplary systems, methods, and computer accessible medium can be provided which can determine information pertaining to a structure based on measured data associated with the structure. For example, the exemplary systems, methods, and computer accessible medium can measure data associated with the structure, transform the measured data into the information using individualized side information, and apply a trust guidance mechanism to resolve ambiguities in the transformed information so as to prevent hallucination or overwriting of the measured data when incorporating the individualized side information. The trust guidance mechanism can comprise incorporating the individualized side information only in an ambiguous space of the inverse problem, and the incorporation can comprise projecting a quantity derived from the individualized side information onto an ambiguous space associated with the inverse problem. The ambiguous space can comprise a vector space associated with singular values below a predetermined threshold of a forward operator of the inverse problem. The transformation can be obtained by a sequence of iterations, each iteration comprising one of a data-consistency update, a trust-guidance update via projection onto the ambiguous space, or a learned refinement update.

The determining of the information pertaining to the structure can include an image reconstruction problem comprising generation of at least one image from the measured data, and the image(s) can include a magnetic resonance (MR) image. The image can be reconstructed from undersampled data, or from accelerated or low-quality data arising from low-cost or accessible image acquisition hardware. The individualized side information can comprise at least one of raw measurement data, reconstructed images, non-image data (including one or more of text, tabular data, and metadata), representations learned from relevant tasks, genetic data, electronic health record data, laboratory results, clinician notes, radiology reports, pathology reports, wearable sensor measurements, or a plurality of multi-modal sources used simultaneously. One or more images of a different contrast may not be registered to the image, and the transformation can comprise a trained learning model with data augmentation simulating misregistration between the individualized side information and the image, so as to reduce the effects of misregistration.

In further exemplary embodiments of the present disclosure, the side information can comprise one or more images of a different contrast from the same imaging session as the target scan, one or more prior images acquired in a different imaging session from the same subject (optionally using a different scanner), one or more images acquired using a different imaging modality, non-image contextual data (e.g., text, tabular data, and/or metadata), or any combination thereof used simultaneously. In still further exemplary embodiments, the learning model can incorporate the side information using a conditional guidance network that depends both on the side information and on a current iterate of the reconstruction, thereby learning implicit alignment and protocol adaptation without requiring a separate pre-registration pipeline. A primary benefit can be that an incomplete scan can be conducted faster and/or with less data collected than is traditionally required for a complete scan, or alternatively that image quality at a given level of acceleration can be improved, thereby enabling use of image acquisition hardware with reduced cost and increased accessibility.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary visual representation of an LIP associated with MR image reconstruction according to an exemplary embodiment of the present disclosure;

FIG. 1B is an exemplary overview of an exemplary trust-guided disambiguation of solutions to the LIP of FIG. 1A according to an exemplary embodiment of the present disclosure;

FIG. 1C is an exemplary visualization of a full end-to-end deep learning framework, according to an exemplary embodiment of the present disclosure;

FIG. 1D is an exemplary illustration of components of each cascade element in the cascade of FIG. 1C, according to an exemplary embodiment of the present disclosure;

FIG. 1E is an exemplary illustration of U-Net architecture used in a refinement block (Φ) and the ASC block (H), according to an exemplary embodiment of the present disclosure;

FIG. 2A is a set of exemplary MR Coronal PDFS knee image reconstructions at 20×acceleration compared with unaccelerated reference image according to an exemplary embodiment of the present disclosure;

FIG. 2B is a set of exemplary MR Axial FLAIR brain image reconstructions at 36×acceleration compared with unaccelerated reference image according to an exemplary embodiment of the present disclosure;

FIG. 3 is a series of exemplary MR image reconstructions of a knee from test K1 showing the effectiveness of exemplary embodiments of the present disclosure;

FIG. 4 is a series of exemplary MR image reconstructions of a knee from test K1 showing the effectiveness of exemplary embodiments of the present disclosure compared to other undersampled methods as well as a full sampling method;

FIG. 5 is a series of exemplary MR image reconstructions of a brain from test B2 showing the effectiveness of exemplary embodiments of the present disclosure at the challenging acceleration level of 45×(15×undersampling and 3×repetition reduction), in comparison to baselines;

FIG. 6 is a set of representative reconstructed images for an ablation study where (1) is reconstruction using an exemplary embodiment of the present disclosure trained with registered side information, encountering registered side information during inference, (2) is reconstruction using an exemplary embodiment of the present disclosure trained with augmentations simulating misregistrations, encountering misregistered side information during inference, (3) is reconstruction from E2E-VarNet without access to side information, and the right image is misregistered side information, according to an exemplary embodiment of the present disclosure;

FIG. 7 is a set of exemplary graphs illustrating exemplary results from the first part of an exemplary ablation study where an exemplary embodiment of the present disclosure, MC-VarNet and E2E-VarNet a shown as a function of Mean SSIM (%) for both main acceleration and side acceleration in accordance with certain exemplary embodiments of the present disclosure;

FIG. 8 is a set of exemplary graphs illustrating exemplary quantitative evaluation results for SSIM, PSNR, and NRMSE over the test dataset for test K3 in accordance with certain exemplary embodiments of the present disclosure;

FIG. 9 is an exemplary set of selected knee image reconstructions from test K1, including DMSI, exemplary embodiments of the present disclosure, exemplary embodiments of the present disclosure with added noise, and a target, in accordance with certain exemplary embodiments of the present disclosure;

FIG. 10 is an illustration of an exemplary block diagram of an exemplary system in accordance with certain exemplary embodiments of the present disclosure;

FIG. 11 is an exemplary overview of a longitudinal trust-guided disambiguation of solutions to the LIP, in which a plurality of axial T2-weighted slices from a most recent prior study of a subject are used as side information to reconstruct a target axial T2-weighted slice in a follow-up exam from highly undersampled k-space measurements, according to an exemplary embodiment of the present disclosure;

FIG. 12 is a set of exemplary axial T2-weighted brain reconstructions at 20×acceleration, comparing reconstructions with and without the use of longitudinal prior images as side information, illustrating recovery of fine neuroanatomical details and pathologies by the exemplary systems, methods, and computer accessible medium according to the exemplary embodiments of the present disclosure; and

FIG. 13 is a set of exemplary axial T2-weighted brain reconstructions at 20×acceleration, demonstrating effectiveness of the exemplary systems, methods, and computer accessible medium according to the exemplary embodiments of the present disclosure in utilizing longitudinal priors relative to matched-capacity baselines that leverage such priors, together with a corresponding undersampling mask, zoomed-in regions, absolute difference maps, and side-information slices acquired in a prior imaging session exhibiting rigid misalignment relative to the current scan, according to the exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the Figures, it is done so in connection with the illustrative embodiments and is not limited by the particular embodiments illustrated in the Figures and the appended claims.

### DETAILED DESCRIPTION OF EXEMPLARY EMBODIMENTS

The following description of exemplary embodiments provides non-limiting representative examples referencing numerals to particularly describe features and teachings of different exemplary aspects and exemplary embodiments of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

Reducing MRI scan times can improve patient care and lower healthcare costs. Various acceleration methods are designed to reconstruct diagnostic-quality images from sparse k-space data, via an ill-posed or ill-conditioned linear inverse problem (LIP). To address the resulting ambiguities, it is crucial to incorporate prior knowledge into the optimization problem, e.g., in the form of regularization.

Another form of prior knowledge less commonly used in medical imaging is the readily available auxiliary data (a.k.a. side information) obtained from sources other than the current acquisition. The exemplary systems, methods, and computer accessible medium according to the exemplary embodiments of the present disclosure present a Trust-Guided Variational Network (TGVN), an end-to-end deep learning framework that can effectively and reliably integrate side information into LIPs (see, e.g., Refs. 64, 65). The effectiveness of the exemplary systems, methods, and computer accessible medium according to the exemplary embodiments of the present disclosure can be demonstrated in multi-coil, multi-contrast MRI reconstruction, where incomplete or low-SNR measurements from one contrast can be used as side information to reconstruct high-quality images of another contrast from heavily under-sampled data. The effectiveness of the exemplary systems, methods, and computer accessible medium according to the exemplary embodiments of the present disclosure can be further demonstrated in longitudinal MRI reconstruction, where one or more images from the most recent prior imaging session of the same subject are used as side information to reconstruct images from heavily undersampled current-session k-space measurements.

Exemplary side information can thus comprise, without any limitation: (i) one or more images of a different contrast acquired in the same imaging session as the target scan; (ii) one or more prior images acquired in one or more different imaging sessions of the same subject, optionally on a same or different scanner; (iii) one or more images acquired using a different imaging modality; and/or (iv) non-image contextual data, including text, tabular data, or metadata; or combinations thereof used simultaneously. In longitudinal settings, e.g., an exemplary longitudinal variant of the disclosure, denoted L-TGVN, can incorporate a dual-encoder, shared-decoder conditional guidance network that jointly processes the longitudinal prior and a current iterate of the reconstruction, thereby learning implicit alignment and protocol adaptation without requiring an explicit pre-registration pipeline, while the trust mechanism constrains the influence of the prior to remain consistent with the acquired current-session measurements so that clinically relevant interval changes (e.g., pathology progression) are preserved rather than suppressed.

TGVN and L-TGVN of the exemplary systems, methods, and computer accessible medium according to the exemplary embodiments of the present disclosure can be robust across different contrasts, anatomies, and field strengths. Compared to baselines utilizing side information, the exemplary systems, methods, and computer accessible medium according to the exemplary embodiments of the present disclosure can achieve superior image quality while preserving subtle pathological features even at challenging acceleration levels, drastically speeding up acquisition while minimizing hallucinations (e.g., generation of image content not supported by the measurement data).

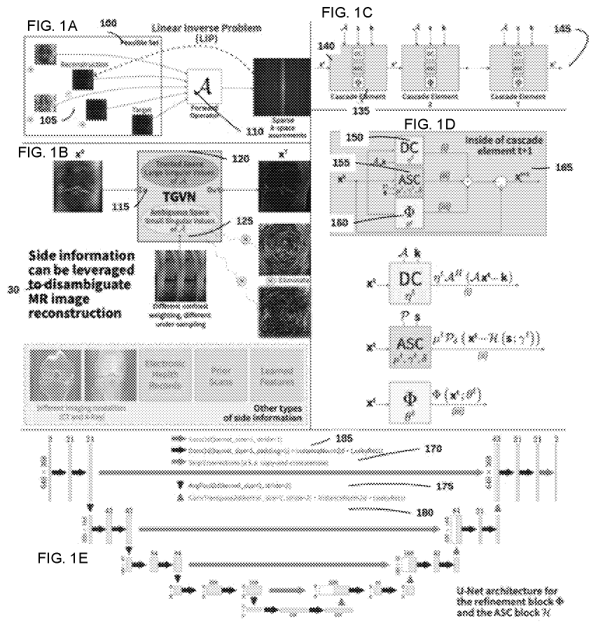

FIGS. 1A-1E show an exemplary end-to-end trainable deep learning approach accordingly to the exemplary systems, methods, and computer accessible medium according to the exemplary embodiments of the present disclosure that can efficiently and reliably integrate side information to solve LIPs. The exemplary systems, methods, and computer accessible medium according to the exemplary embodiments of the present disclosure can use the side information only to disambiguate the subspace of solutions that the forward operator cannot reliably distinguish based on the measured data (i.e., the ambiguous space).

Specifically, FIG. 1A shows an exemplary visual representation of an LIP associated with MR image reconstruction 105 providing a feasible set 100 of possible solutions according to an exemplary embodiment of the present disclosure. FIG. 1B illustrates an exemplary overview of trust-guided disambiguation of solutions to the LIP of FIG. 1A according to an exemplary embodiment of the present disclosure. As shown in FIG. 1B, a TGVN processing block 115 can receive undersampled k-space measurements and side information input 130 and can decompose the solution space into a trusted space 120, corresponding to components that can be reliably determined from the acquired measurements, and an ambiguous space 125, corresponding to components that cannot be reliably distinguished based on the measured data alone. The TGVN processing block 115 can use the side information input 130 preferentially in the ambiguous space 125 to disambiguate the solution while preserving data consistency in the trusted space 120. FIG. 1C shows an exemplary visualization of a full end-to-end deep learning framework according to an exemplary embodiment of the present disclosure, in which a plurality of T cascade elements 135 are connected in series. An initial estimate input 140, derived from the undersampled k-space measurements (e.g., via the adjoint of the forward operator), can be provided to the first cascade element 135, and each successive cascade element 135 can iteratively refine the reconstruction to produce a final reconstruction output 145. FIG. 1D provides an exemplary illustration of components of each cascade element 135 in the cascade of FIG. 1C, the components including data consistency (DC) block 150 parametrized by η, ambiguous space consistency (ASC) block 155 parametrized by μ, γ, δ, and refinement block 160 (Φ) parametrized by θ, along with their inputs, their outputs, and the final aggregation of outputs via aggregation output 165 according to an exemplary embodiment of the present disclosure. FIG. 1E provides an exemplary illustration of U-Net architecture used in the refinement block 160 (Φ) and the ASC block 155 (H), illustrated for a 640×368 coil-combined image, with 4 pooling levels and an initial 21 channels in the first double-convolution, doubling at each pooling level, and including skip connections 170, average pooling layers 175, transposed convolution layers 180, and instance normalization and LeakyReLU activation 185 according to an exemplary embodiment of the present disclosure.

For example, the exemplary systems, methods, and computer accessible medium according to the exemplary embodiments of the present disclosure can introduce a learnable squared Euclidean distance constraint, termed the ambiguous space consistency constraint, into the regularized least-squares reconstruction formulation of the LIP to reduce and/or eliminate undesirable solutions from the ambiguous space of the forward operator. This ambiguous space consistency constraint can be seamlessly integrated into any deep unrolled network. The exemplary systems, methods, and computer accessible medium according to the exemplary embodiments of the present disclosure can be trained end-to-end with full supervision to maximize a similarity metric between the reconstructed and the ground-truth image, requiring minimal modifications to integrate the constraint.

The effectiveness of the exemplary systems, methods, and computer accessible medium according to the exemplary embodiments of the present disclosure can be demonstrated in the challenging domain of multi-coil, multi-contrast MR image reconstruction, where incomplete or low-SNR measurements from complementary contrast weighting can be used as side information to reconstruct images with a different target contrast from exceedingly small quantities of k-space measurements (on the order of 20×undersampling in a single phase-encoding direction) across different anatomies and field strengths. Compared to recently proposed DL-based solutions, the exemplary systems, methods, and computer accessible medium according to the exemplary embodiments of the present disclosure can leverage side information more efficiently and reliably, preserving fine anatomical and pathological details obliterated by other methods at high acceleration levels, and can achieve statistically significant improvements in reconstruction performance while also being robust to degradations in the quality of side information.

To summarize:
i. The exemplary systems, methods, and computer accessible medium according to the exemplary embodiments of the present disclosure can leverage side information to reliably solve ill-posed or ill-conditioned LIPs.
ii. The effectiveness of the exemplary systems, methods, and computer accessible medium according to the exemplary embodiments of the present disclosure can be demonstrated in multi-coil multi-contrast MR image reconstruction, using incomplete or low-SNR measurements from complementary contrast weighting as side information.
iii. The robustness of the exemplary systems, methods, and computer accessible medium according to the exemplary embodiments of the present disclosure can be demonstrated by showing its efficacy for different contrasts across multiple anatomies and multiple field strengths. The results of various ablation studies are reported to illuminate the origins of this robustness.
iv. The exemplary systems, methods, and computer accessible medium according to the exemplary embodiments of the present disclosure can be shown to leverage side information more efficiently than other recent ML-based solutions, achieving statistically significant improvements in image reconstruction performance and pushing the boundaries of current techniques in medical imaging and beyond.

### Exemplary Information

### Exemplary Multi-Coil MR Image Acquisition

In MR imaging, measurements can be acquired in the spatial frequency (e.g., k-space) domain, and the measurements are related to the estimated MR image through the linear forward operator A. These measurements can be grouped into a complex-valued vector \tilde{k}, and the elements of \tilde{k} represent Fourier coefficients of the structure of the continuous object being imaged. Specifically, the exemplary systems, methods, and computer accessible medium according to the exemplary embodiments of the present disclosure can define a discrete estimated MR image x, such that \tilde{k}=F(x)+ϵ, where ϵ is complex Gaussian noise and F denotes the Fourier transform operator. The vector x∈C^{MN} is a complex vector of size MN, where M and N are pixel dimensions of the two-dimensional image being sought.

In parallel imaging (PI), the scanner captures multiple views of the anatomy modulated by the sensitivities Sᵢ of the receiver coils, which can be represented by diagonal matrices Sᵢ∈C^{MN×MN}. In this case the relationship becomes \tilde{k}ᵢ=F(Sᵢx)+ϵᵢ, for each i∈1, 2 . . . , N_{c}, where N_{c} denotes the number of coils. To simplify notation, the k-space data from all coils can be aggregated into a single tensor \tilde{k}=(\tilde{k}₁, . . . , \tilde{k}_{N}_{_{c}}) and can define the expand operator (E) which maps the complex image to multi-coil k-space. That is, E:x [symbol] (F(S₁x), . . . , F(S_{N}_{_{c}}x)). To accelerate MR acquisition, fewer k-space samples can be acquired, which can be denoted by a binary diagonal mask M∈{0,1}^{MN×MN}. Thus, the set of under-sampled k-space measurements can be denoted as k [symbol] M\tilde{k}=(M\tilde{k}₁, . . . , M\tilde{k}_{N}_{_{c}}), and the forward operator A mapping the underlying image to the under-sampled and noisy k-space measurements in multi-coil MR image acquisition is equal to M∘E. That is,

k= [symbol] x+ϵ′=( [symbol] ∘ε)x+ϵ′, (1)

where ϵ′ denotes the complex Gaussian noise in the under-sampled k-space measurements.

### Exemplary Deep Learning for Parallel MR Image Reconstruction

Given the forward operator A and the k-space data k, estimating x can be considered a well-posed problem if it meets the following three criteria (called the Hadamard conditions): 1) existence of a solution, 2) uniqueness of the solution, and 3) stability of the solution. (See, e.g., Ref. 19). Accelerated parallel MR image reconstruction, however, like most real-word problems, is either ill-posed, failing to meet one or more of these criteria, or ill-conditioned, with small errors in the measurements leading to much larger errors in the image estimate x. This can be because the sparse set of measurements k makes the above system of equations (1) either underdetermined, with a potentially infinite set of solutions, or ill-conditioned, with a large yet finite condition number. When the measurement noise is Gaussian, the maximum likelihood estimate of a solution to (1) is given by

$$\hat{x} = arg {min}_{x} \frac{1}{2} {‖ Ax - k ‖}_{2}^{2} .$$

To address its not-well-posed or not-well-conditioned nature, the LIP can be reformulated to impose additional constraints or requirements on the solution. By incorporating appropriate additional constraints, a reliable approximate solution can be derived. More formally, e.g., let Ψ(⋅) denote a regularization function that imposes certain constraints on the possible solutions x, e.g., sparsity in the wavelet or total variation domain. Then, the optimization problem can be modified as follows:

$$\hat{x} = arg {min}_{x} \frac{1}{2} {‖ 𝒜 x - k ‖}_{2}^{2} + Ψ ( x ) . \qquad ( 2 )$$

In deep-learning based unrolled networks, such as, e.g., the End-to-end Variational Network (E2E-VarNet) (see, e.g., Ref. 6), one learns a regularization function from the training data to maximize a desired similarity metric between the reconstructed image \hat{x} and the ground truth. Specifically, E2E-VarNet starts with an initial estimate x⁰ of the solution to Ax=k, and uses the Landweber method (see, e.g., Ref. 20) to iteratively refine its estimate. Furthermore, it replaces the gradient of the regularization function Ψ(x) with a neural network Φ, parametrized by θ^{t} at each iteration t. More formally, E2E-VarNet executes the following sequence of steps for a total of T iterations (implemented in T cascade elements similar to FIG. 1), starting with x⁰=A^{H}k:

x^{t+1}=x^{t}−η^{t} [symbol] ^{H}( [symbol] x^{t}−k)−Φ(x^{t};θ^{t}), (3)

where A^{H}=E^{H} ∘ M is the Hermitian adjoint of A. The second term on the right hand side is usually referred to as data consistency, as it guides x to be maximally consistent with the acquired measurements. At the end of iteration T, x^{T} parameterized by Θ=θ⁰, . . . , θ^{T-1}, η⁰, . . . , η^{T-1} can be obtained. Assuming access to ground truth x*, parameters Θ can be learned in a supervised manner to maximize a desired similarity between x^{T} and x*.

### Exemplary Related Work

Described below, is how prior work has utilized side information in MR image reconstruction. While side information can take various forms, most studies have focused on complementary contrast information—reconstructing the target contrast by leveraging information from other contrast(s). This approach differs from both single-contrast and joint multi-contrast reconstruction.

### Exemplary Initial Attempts

The use of side information in medical image reconstruction dates back to at least the 1990s. Ref. 21 demonstrated tomographic image reconstruction based on a weighted Gibbs penalty, where the weights are determined by anatomical boundaries in high-resolution MR images. Ref. 22 suggested a Bayesian method, whereby maximum a posteriori (MAP) estimates of PET and SPECT images may be reconstructed with the aid of prior information derived from registered anatomical MR images of the same slice. Some of the earlier attempts also utilized handcrafted priors. (See, e.g., Refs. 13-15 and 23-29). Later, dictionary-learning-based methods were proposed. (See, e.g., Refs. 16 and 30).

### Exemplary End-to-End Methods

More recently, multiple authors have proposed end-to-end deep learning-based models that leverage side information for MR image reconstruction. Specifically, Refs. 31 and 32 proposed combining T1-weighted images and under-sampled T2-weighted images to reconstruct fully sampled T2-weighted images using a Dense U-Net model. Ref. 17 introduced a Dilated Residual Dense Network (DuDoRNet) for dual domain restorations from under-sampled MRI data to simultaneously recover k-space and images. Ref. 33 developed a multi-modal transformer (‘MTrans’) for accelerated MR imaging which transferred multi-scale features from the target modality to the auxiliary modality. Rather than manually designing fusion rules, Ref. 18 presented a multi-contrast VarNet (‘MC-VarNet’) to explicitly model the relationship between different contrasts.

### Exemplary Generative Models

Generative models utilizing side information for MR image reconstruction are GAN-based and score-based algorithms. These models can be divided into reconstruction and synthesis methods, in which the former is the focus of the exemplary systems, methods, and computer accessible medium according to the exemplary embodiments of the present disclosure. Specifically, Ref. 34 utilized conditional GANs with three priors—shared high-frequency, low-frequency, and perceptual priors. Ref. 35 proposed a framework for estimating objects from incomplete imaging measurements by optimizing in the latent space of a style-based generative model, using constraints from a related prior image. Ref. 36 introduced a score-based generative model (‘DMSI’) to learn a joint Bayesian prior over multi-contrast data.

### Exemplary Range-Null Space Decomposition

Range-null space decomposition can involve breaking down a vector space into two orthogonal subspaces: the range (or column space) and the null space of a linear operator. Given a linear operator A, the range space consists of all possible outputs of A, while the null space contains all vectors that are mapped to zero by A. This decomposition can be particularly useful in solving LIPs, as it can allow for the separation of components that are preserved by the operator from those that are not. The combination of range-null space decomposition with deep learning was first explored in Ref. 37. More recently, Ref. 38 introduced a GAN-based super-resolution method that learns only the null-space component while fixing the range-space component; Ref. 39 presented a diffusion model for LIPs which refines only the null-space contents during the reverse diffusion process; and Ref. 40 addressed the inconsistencies in MR image reconstruction by decomposing input data and selectively feeding the null-space component into proximal mapping.

Despite notable advancements, existing methods that incorporate side information for solving LIPs still struggle with highly under-sampled data, often producing reconstructions with degraded image quality and/or hallucinations. The former can be attributed to a lack of efficiency in exploiting side information (i.e., insufficient ability to disambiguate the solution space), while the latter can represent over-reliance on it. Consequently, harnessing the full potential of side information while mitigating hallucinations remains an open problem that can have a transformative impact on the efficiency and accessibility of medical imaging.

In longitudinal settings, several works have studied serial imaging where a baseline scan provides subject-specific context for reconstructing a follow-up scan from undersampled measurements, often by promoting sparsity in the baseline-follow-up difference or using prior-weighted regularization (see, e.g., Refs. 29, 57, and 59). A prominent line of work formulated longitudinal reconstruction through reference-based compressed sensing with mechanisms that adaptively reduce reliance on the prior when longitudinal similarity breaks down (see, e.g., Refs. 13 and 14). Related optimization-based formulations have also been explored, combining data fidelity with sparsity and explicit penalties on deviation from a reference image (see, e.g., Ref. 58). More recently, learning-based longitudinal reconstruction approaches have emerged that aim to reduce reliance on paired training data and pipelines that learn to align and fuse prior information (see, e.g., Refs. 60-62). Despite substantial progress, existing longitudinal methods often face a core tradeoff: tightly prior-coupled approaches can overfit to outdated anatomy or pathology, while looser reliance on priors may underutilize subject-specific information at challenging levels of acceleration, where explicit image-domain registration can also become unreliable.

The exemplary systems, methods, and computer accessible medium according to the exemplary embodiments of the present disclosure address this challenge by constraining the influence of longitudinal priors to remain consistent with the acquired measurements, providing a principled trust mechanism that guards against over-reliance while still exploiting subject-specific context, and by avoiding explicit pre-registration between visits through feature-space fusion within the trust-guidance module, thereby accommodating cross-visit acquisition changes as well.

### Exemplary Trust Guided Variational Network (TGVN) and Longitudinal Trust Guided Variational Network (L-TGVN)

The exemplary systems, methods, and computer accessible medium according to the exemplary embodiments of the present disclosure can effectively and reliably leverage side information to impose additional constraints into the LIP and guide the solution to fall within a contextually appropriate distribution. In this setting, it can be assumed that the exemplary systems, methods, and computer accessible medium according to the exemplary embodiments of the present disclosure can assume have access to the additional side information denoted by s when solving for x using the system of equations Ax=k. As long as s and x are conditionally dependent given k (i.e., the conditional mutual information I(s; x|k)>0), the knowledge of s can be exploited to reduce the uncertainty in estimating x from k. (See, e.g., Ref. 41). As such, the solution provided by the exemplary systems, methods, and computer accessible medium according to the exemplary embodiments of the present disclosure can assume the existence of such conditional dependence.

### Exemplary Motivation: Ambiguous Space Consistency

Deep learning and physics-based unrolled networks have shown notable success in MR image reconstruction from sparse k-space data (see, e.g., Ref. 42), primarily due to their ability to enforce data consistency—ensuring that the reconstructed images closely match the acquired measurements. However, while data consistency is crucial for aligning the solution with the observed data, it might not be sufficient to resolve inherent ambiguities in the solution space, particularly at higher accelerations where an abrupt degradation in image quality has been highlighted (see, e.g., Ref. 12), rendering the images non-diagnostic.

To address this issue, the exemplary systems, methods, and computer accessible medium according to the exemplary embodiments of the present disclosure introduce the concept of ambiguous space consistency, which can go beyond data consistency and can complement it. Essentially, the exemplary systems, methods, and computer accessible medium according to the exemplary embodiments of the present disclosure can identify a subspace of images that could significantly alter reconstruction quality without substantially affecting the data inconsistency loss

$$- {‖ Ax - k ‖}_{2}^{2} -$$

of the MR image reconstruction problem. The exemplary systems, methods, and computer accessible medium according to the exemplary embodiments of the present disclosure can then use side information preferentially in this ambiguous space to resolve ambiguities without compromising information that has been encoded reliably in measured data. Conceptually, one might expect the ambiguous space to be associated with low singular values of the forward problem, since it is known that singular values are largest in the reverse mapping where they were smallest in the forward mapping, resulting in amplified noise and increased sensitivity to small perturbations.

Let xₚ be a particular solution to the equation

$$Ax = k \sum and let {\sum }_{i} {σ}_{i} {u}_{i} {v}_{i}^{H}$$

denote the singular value decomposition (SVD) of A. Given a small positive threshold δ, the exemplary systems, methods, and computer accessible medium according to the exemplary embodiments of the present disclosure can define the ambiguous space as the subspace spanned by the right singular vectors vᵢ with corresponding singular values σᵢ smaller than δ, and can denote it as W_{δ}(A). Observe that if any unit vector xₐ∈W_{δ}(A) to xₚ is added, the data inconsistency loss

$${‖ A ( {x}_{p} + {x}_{a} ) - k ‖}_{2}^{2}$$

can at most be δ². In other words, perturbing a solution that aligns with the observed measurements by adding a vector from the ambiguous space can result in only a minor change to the objective value. In the exemplary systems, methods, and computer accessible medium according to the exemplary embodiments of the present disclosure, perturbation in the ambiguous space can create only minor data inconsistency. However, only certain xₐ maximize the desired similarity between xₚ+xₐ and x*, indicating that, once a particular solution is found, images from W_{δ}(A) can introduce ambiguity in the reconstruction problem. That is, they might visually alter the reconstruction quality without significantly affecting the data inconsistency loss

$${‖ Ax - k ‖}_{2}^{2} .$$

Inspired by this observation, the exemplary systems, methods, and computer accessible medium according to the exemplary embodiments of the present disclosure can explicitly learn a constraint that removes undesirable solutions from W_{δ}(A). The exemplary systems, methods, and computer accessible medium according to the exemplary embodiments of the present disclosure can project x onto W_{δ}(A) with the orthogonal projector P_{δ} to obtain xₐ, and to guide xₐ to be maximally consistent with the side information s using a learnable module H parametrized by γ. Specifically, the exemplary systems, methods, and computer accessible medium according to the exemplary embodiments of the present disclosure can add a squared Euclidean distance constraint

$${‖ {P}_{δ} x - H ( s ; γ ) ‖}_{2}^{2}$$

to (2) to obtain

$$\hat{x} = arg {min}_{x} \frac{1}{2} {‖ 𝒜 x - k ‖}_{2}^{2} + \frac{β}{2} {‖ {𝒫}_{δ} x - ℋ ( s ; γ ) ‖}_{2}^{2} + Ψ ( x ) . \qquad ( 4 )$$

Ambiguous space consistency (ASC) is both analogous to and complementary to data consistency (DC). Both are linear in x, and both ensure that a linear transformation applied to x is close to a desired vector—acquired measurements in DC and a learned transformation of side information in ASC. This linearity helps to maintain the simplicity of Landweber iterations, since the gradient of the ASC with respect to x is simply P_{δ}(x−H(s; γ)). By design, however, ASC operates in the ambiguous space. As a result, it does not adversely affect DC, thereby minimizing the risk of hallucinations resulting from over-reliance on side information. This is a crucial distinction between the exemplary systems, methods, and computer accessible medium according to the exemplary embodiments of the present disclosure and other methods.

TABLE 1
Under sampling, in either a random or a uniform pattern, was performed along the
phase-encoding (PE) direction. Center frequency (CF) is the portion of fully sampled central
PE lines. For knee tests (K1, K2, and K3), acceleration was achieved solely by under
sampling. For brain tests (B1 and B2), the total acceleration factors include both under
sampling (18×and 15x for main information) and reduction in the number of repetitions (2×
and 3× for main, 3× for side information).

| Test | Contrast | Acceleration | CF | Contrast | Acceleration | CF |
|---|---|---|---|---|---|---|
| K1 | PDFSw | Random-20× | 3 | PDw | Uniform-2× | 0 |
| K2 | PDFSw | Random-14× | 3 | PDw | Uniform-3× | 0 |
| K3 | PDFSw | Random-6× | 5 | PDw | Uniform-3× | 0 |
| B1 | FLAIR | Uniform-36× | 2 | T2w | 3× | — |
| B2 | T1w | Uniform-45× | 2 | T2w | 3× | — |

One reason for selecting a more general projector P_{δ} rather than simply using an orthogonal projector onto the null space of A can be twofold. First, in practice, the forward operator matrix A in parallel MR imaging and other LIPs can have a trivial null space but still exhibit many small, non-zero singular values. This is the reason for high noise amplification at higher acceleration rates. (See, e.g., Ref. 43). Second, even when the null space is non-trivial (i.e., it does not only contain the zero vector), the presence of small singular values can pose challenges, and the proposed approach can further assist in resolving these ambiguities. In the limit as δ approaches 0 from above, the set W_{δ}(A) converges to the null space of A (i.e., lim_{δ↓0}W_{δ}(A)=N(A)) and the projection P_{δ} approaches the orthogonal projector onto N(A). Hence, the exemplary systems, methods, and computer accessible medium according to the exemplary embodiments of the present disclosure can be more general than simply resolving the null space—it can naturally encompass null space resolution as a special case.

### Exemplary Solution: Iterative Optimization

An exemplary solution to (4) can be obtained using a cascade of neural networks similar to those used in the E2E-VarNet method. As the added constraint involves only a squared Euclidean distance, its integration into (3) can be straightforward. Starting with x₀=A^{H}k, the exemplary systems, methods, and computer accessible medium according to the exemplary embodiments of the present disclosure can execute the following sequence of steps for a total of T iterations.

$${x}^{t + 1} = {x}^{t} - {η}^{t} {𝒜}^{H} ( 𝒜 {x}^{t} - k ) - {\underbrace{{μ}^{t} {𝒫}_{δ} ( {x}^{t} - H ( s ; {γ}^{t} ) )}}_{trust - guidance} - Φ ( {x}^{t} ; {θ}^{t} ) . \qquad ( 5 )$$

At the end of iteration T, the exemplary systems, methods, and computer accessible medium according to the exemplary embodiments of the present disclosure can obtain x^{T} parameterized by Ω≙Θ∪δ, γ⁰, . . . , γ^{T-1}, μ⁰, . . . , μ^{T-1}. Assuming access to ground truth x*, the parameters Ω can be learned in a supervised manner to maximize a desired similarity between x^{T} and x*. It is worth noting that the parameter δ can be learned from the data as proposed, or it can be fixed based on the coil specifications and undersampling pattern by analyzing the distribution of singular values.

In high-dimensional problems like parallel MR imaging, the computational burden of working directly with large-scale operators can be prohibitive. Therefore, instead of explicitly calculating the SVD of the forward operator, which would be computationally expensive, the exemplary systems, methods, and computer accessible medium according to the exemplary embodiments of the present disclosure can provide an efficient alternative. Here, the exemplary systems, methods, and computer accessible medium according to the exemplary embodiments of the present disclosure can present an efficient approximation of the exact orthogonal projector P_{δ}, which bypasses the need for SVD computation.

This exemplary approach can be crucial for managing the scale of the forward operator, which may contain hundreds of thousands of rows and columns, making explicit methods infeasible. For a set K, let 1_{K}(x) denote an indicator function that equals 1 if x∈K and 0 otherwise. Given δ, the exact projector can be written as

$${P}_{δ} = {\sum }_{i} {1}_{[ 0 , δ )} ( {σ}_{i} ) {v}_{i} {v}_{i}^{H} .$$

In lieu of assigning binary weights to the ith projection, the exemplary systems, methods, and computer accessible medium according to the exemplary embodiments of the present disclosure can weight them by δ²/(δ²+σ), and define

$${𝒫}_{δ}^{′} {=}^{Δ} {\sum }_{i} \frac{{δ}^{2}}{{δ}^{2} + {σ}_{i}^{2}} {v}_{i} {v}_{i}^{H} = {( I + \frac{1}{{δ}^{2}} {𝒜}^{H} 𝒜 )}^{- 1} , \qquad ( 6 )$$

where I:x [symbol] x denotes the identity operator. The exemplary systems, methods, and computer accessible medium according to the exemplary embodiments of the present disclosure can then calculate the approximate trust-guidance term

$${{μ}^{t} ( l + \frac{1}{{δ}^{2}} {𝒜}^{H} 𝒜 )}^{- 1} ( {x}^{t} - ℋ ( s ; {γ}^{t} ) ) , \qquad ( 7 )$$

efficiently using a small number of conjugate gradient iterations. (See, e.g., Ref. 44).

In further exemplary embodiments of the present disclosure, directed to longitudinal reconstruction where the side information s comprises one or more images acquired in a prior imaging session of the same subject, the residual term inside the trust-guidance projection can depend on both the longitudinal prior and the current iterate of the reconstruction. For example, in an exemplary longitudinal variant denoted L-TGVN, the trust-guidance module can be implemented as a conditional guidance network H(s, x^{t}; γ^{t}) that produces a proposal image conditioned on the current iterate x^{t}, such that the residual before projection is r^{t}=x^{t}−H(s, x^{t}; γ^{t}). In an exemplary implementation, H is a dual-encoder, shared-decoder U-Net: one encoder processes the prior input s, represented as a small stack of neighboring slices from a most relevant prior series selected based on metadata (e.g., z-axis slice position), while a second encoder processes the complex-valued current iterate x^{t} mapped to two real channels; at each resolution, features from the two encoders are fused (e.g., concatenation followed by a 1×1 convolution) to form shared skip connections for a single decoder, which outputs a complex-valued proposal with the same spatial support as the current reconstruction. Conditioning on the current iterate can facilitate the exemplary systems, methods, and computer accessible medium according to the exemplary embodiments of the present disclosure to learn implicit alignment and protocol adaptation, thereby avoiding a separate pre-registration pipeline and accommodating protocol drift and scanner differences between the prior and current sessions. The longitudinal variant can be formulated as an iterative optimization starting from the zero-filled reconstruction and unrolled for T iterations.

Most or all trainable parameters across iterations can be learned end-to-end, e.g., by maximizing a chosen similarity measure (such as structural similarity index, SSIM) between the final iterate x^{T} and a ground-truth target image x*. In still further exemplary embodiments, the side information can comprise multiple series from a given prior study (e.g., T2-weighted and FLAIR), or images from multiple prior imaging sessions, and the dual-encoder structure can be extended to process each additional source in parallel with feature-level fusion.

It is possible to build intuition about ambiguous space consistency by considering a simplified example. For the special case of a single-coil acquisition with a binary under sampling mask M, the approximate projector onto the ambiguous space is given by

$${P}_{δ}^{′} = {{F}^{H} ( \frac{M}{{δ}^{2}} + I )}^{- 1} F .$$

### Here,

$${P}_{δ}^{′} x$$

first transforms the complex-valued image x into k-space. In the k-space domain, the acquired lines are scaled by

$$\frac{{δ}^{2}}{1 + {δ}^{2}} << 1 ,$$

while the non-acquired lines retain unit weight. The weighted k-space can then be transformed back into image space by F^{H}. Since δ is small, the acquired lines are significantly attenuated, causing the non-acquired (masked) lines to dominate the projection. For

$$δ = \frac{1}{3} ,$$

for example, acquired lines are scaled by a factor of 0.1, while the masked lines remain unscaled. In this case, side information can contribute principally to the non-acquired k-space lines while still retaining a nonzero contribution to the acquired lines. In the limit δ↓0, side information contributes only to the non-acquired k-space lines. It should be noted that in the multi-coil setting, coil sensitivities can play a role in spatial encoding, and the distinction between acquired and non-acquired lines can be less clear, but the same general principles apply to the separation of trusted from untrusted components.

### Exemplary Empirical Validation

The efficacy of the approach outlined in the exemplary systems, methods, and computer accessible medium according to the exemplary embodiments of the present disclosure can be validated by using it for multi-coil MR image reconstruction from different contrasts across different anatomies and field strengths, as detailed in Table 1. In all validation tests, the exemplary systems, methods, and computer accessible medium according to the exemplary embodiments of the present disclosure utilized the efficient approximate projection introduced in (6). The empirical validation sought answers to the following four questions:
Q1. Is there any benefit in using the side information?
Q2. How effective is TGVN at utilizing the side information?
Q3. Does projecting onto the ambiguous space provide any benefits compared to no projection?
Q4. How robust is the proposed approach to different undersampling factors, to misregistration between images used for main and side information, and to degradation in the quality of side information?

To answer Q1, the exemplary reconstruction performance of the exemplary systems, methods, and computer accessible medium according to the exemplary embodiments of the present disclosure was compared against an E2E-VarNet of the same capacity that does not utilize side information. Q2 was answered by comparing the performance of the exemplary systems, methods, and computer accessible medium according to the exemplary embodiments of the present disclosure against several recent DL baselines that also leverage side information in image reconstruction: MTrans (see, e.g., Ref. 33), MC-VarNet (see, e.g., Ref. 18), and DMSI (see, e.g., Ref. 36). To address Q3, the performance of the exemplary systems, methods, and computer accessible medium according to the exemplary embodiments of the present disclosure was compared with and without the projection. Q4 was answered by conducting tests using multiple under sampling factors in main and side information, by deliberately introducing misregistrations, and by using irrelevant side information in the form of random samples from a Gaussian distribution. Exemplary findings are provided below.

In these tests, undersampling for both the main and side information was implemented along the phase-encoding direction. The target images can be selected as the root-sum-of-squares (RSS) combination \sqrt{Σᵢ|xᵢ|²} of fully sampled component coil images xᵢ. The exemplary systems, methods, and computer accessible medium according to the exemplary embodiments of the present disclosure can evaluate the reconstruction quality using three metrics: the structural similarity index (see, e.g., Ref. 45) (SSIM, in %), peak signal-to-noise ratio (PSNR, in dB), and normalized root-mean-squared error (NRMSE). Additional training and evaluation details are provided below. To demonstrate the statistical significance of the improvements in image reconstruction metrics, the exemplary systems, methods, and computer accessible medium according to the exemplary embodiments of the present disclosure can perform a Wilcoxon signed-rank test (see, e.g., Ref. 46) between the metrics calculated on the test dataset for TGVN (sₒᵤᵣₛ) as compared to the best-performing baseline (s_{base}, the baseline with the best average score).

In further exemplary empirical validation focused on longitudinal side information, an additional set of tests (LB1 through LB3 described herein, corresponding to 20×, 15×, and 10×net acceleration, respectively) was performed on an in-house, IRB-approved and de-identified fully sampled multi-coil brain k-space dataset comprising 757 training, 91 validation, and 81 test axial T2-weighted (T2w) volumes acquired on 3T and 1.5T scanners using turbo spin-echo sequences. For each subject, a recent prior study was retrieved from a Picture Archiving and Communication System (PACS) and the axial T2w DICOM series of the prior study was used as longitudinal side information, with a median inter-scan interval of 326 days (Q1-Q3: 159-548 days). The dataset is heterogeneous in acquisition parameters, scanner models, and field strengths between the current and prior scans, and exhibits appreciable inter-scan misalignment including in-plane and out-of-plane rotations and translations.

In these longitudinal tests, the exemplary systems, methods, and computer accessible medium according to the exemplary embodiments of the present disclosure were compared against three matched-capacity baselines (approximately 500 million trainable parameters each): (i) E2E-VarNet (see, e.g., Ref. 6) with an expanded learned regularizer, which does not use longitudinal priors; (ii) MTrans (see, e.g., Ref. 33) and (iii) DMSI (see, e.g., Ref. 36), both adapted to longitudinal imaging by using the prior scan as the conditioning input. All longitudinal methods used 11 neighboring slices from the most relevant prior series (selected based on z-axis metadata) as side information to reconstruct each target slice from retrospectively undersampled k-space with random phase-encoding masks and fully-sampled center fractions of 2.5%, 3.3%, and 5% for LB1, LB2, and LB3, respectively. L-TGVN, MTrans, and E2E-VarNet were trained on 8×A100 GPUs (80 GB VRAM) with an equivalent batch size of 64 for 20,000 gradient steps, with the best-validation-SSIM checkpoint selected for testing; DMSI was trained for 70,000 gradient steps with best-validation EDM loss used for checkpoint selection. Test-set evaluation was performed at the patient level (n=81) using SSIM, PSNR, and NRMSE. Statistical significance of improvements was assessed using Wilcoxon signed-rank tests at a 1% significance level.

In knee tests (K1, K2, K3), the exemplary systems, methods, and computer accessible medium according to the exemplary embodiments of the present disclosure can utilize a subset of the multi-coil track of the fastMRI knee dataset—an open-source dataset consisting of k-space measurements from clinical 3T and 1.5T scanners paired with the ground-truth clinical cross-sectional images (see, e.g., Ref. 47). The dataset can comprise coronal MR scans of e.g., 428 patients using a proton-density weighting with fat suppression (PDFSw) and proton density weighting without fat suppression (PDw). Data acquisition can employ an e.g., 15-channel knee coil array and Cartesian 2D Turbo Spin Echo (TSE) pulse sequences. The exemplary systems, methods, and computer accessible medium according to the exemplary embodiments of the present disclosure can split the dataset into three non-overlapping subsets of e.g., sizes 368, 30, and 30 image volumes, for training, validation, and test sets, respectively, with a total of e.g., 15,231 slices. It should be noted that pathology labels were available for this dataset (see, e.g, Ref. 48); even the most prevalent pathology—meniscus tear-appears in only e.g., 2,163 out of 15,231 slices, highlighting the sparsity of pathological features at the slice level. Reconstruction performance of the exemplary systems, methods, and computer accessible medium according to the exemplary embodiments of the present disclosure can be visually assessed by comparing slices exhibiting pathology (verified by an experienced musculoskeletal radiologist) during inference.

Brain tests (B1, B2) can utilize e.g., the M4Raw dataset (see, e.g., Ref. 49)—a publicly available multi-channel k-space dataset of brain scans of 183 healthy volunteers acquired using a low-field (0.3T) scanner. It includes axial MR scans with three contrasts, acquired using a 4-channel array: T1-weighted (T1w), T2-weighted (T2w), and fluid-attenuated inversion recovery (FLAIR). Each scan has 18 slices per contrast with varying numbers of repetitions. The exemplary systems, methods, and computer accessible medium according to the exemplary embodiments of the present disclosure can use single-repetition measurements to reconstruct images with quality similar to that of multi-repetition aggregated RSS targets. The training, validation, and test sets can include e.g., 128, 30, and 25 volumes, respectively. In both knee and brain tests, the exemplary systems, methods, and computer accessible medium according to the exemplary embodiments of the present disclosure can ensure that patient-level and study-level splits are used, so that any single patient's data appears exclusively in the training, the validation, or the test set only. Details regarding test settings, including contrast and acceleration details, are provided in Table 1.

TABLE 2
Exemplary quantitative evaluation results - SSIM, PSNR, and NRMSE are shown
for knee (K1, K2, K3) and brain (B1, B2) tests using the exemplary systems, methods, and
computer accessible medium according to the exemplary embodiments of the present
disclosure and baselines. For each evaluation metric and each reconstruction method, the
mean and standard error of the mean over the test dataset are reported. Bold-face values
indicate the best performance in each category, which in all cases is achieved by
reconstruction of the exemplary systems, methods, and computer accessible medium
according to the exemplary embodiments of the present disclosure. For SSIM and PSNR.
higher is better; for NRMSE, lower is better.

| Metric | Test | TGVN | DMSI | MC-VarNet | MTrans | E2E-VarNet |
|---|---|---|---|---|---|---|
| SSIM | K1 | 84.92 ± 0.19 | 56.99 ± 0.31 | 82.89 ± 0.21 | 80.84 ± 0.23 | 81.33 ± 0.23 |
| | K2 | 85.52 ± 0.19 | 58.76 ± 0.31 | 83.13 ± 0.21 | 81.25 ± 0.22 | 83.40 ± 0.21 |
| | K3 | 88.02 ± 0.17 | 64.35 ± 0.31 | 86.47 ± 0.18 | 85.29 ± 0.19 | 87.42 ± 0.17 |
| | B1 | 87.34 ± 0.12 | — | 86.95 ± 0.12 | 84.03 ± 0.14 | 81.40 ± 0.15 |
| | B2 | 89.34 ± 0.10 | — | 88.66 ± 0.12 | 85.72 ± 0.17 | 86.11 ± 0.13 |
| PSNR | K1 | 30.92 ± 0.07 | 22.22 ± 0.10 | 29.97 ± 0.07 | 28.93 ± 0.07 | 29.30 ± 0.07 |
| | K2 | 31.31 ± 0.07 | 22.68 ± 0.10 | 30.07 ± 0.07 | 29.11 ± 0.07 | 30.37 ± 0.07 |
| | K3 | 32.89 ± 0.08 | 24.24 ± 0.10 | 31.95 ± 0.07 | 31.26 ± 0.07 | 32.59 ± 0.07 |
| | B1 | 30.81 ± 0.08 | — | 30.75 ± 0.08 | 28.70 ± 0.08 | 27.14 ± 0.08 |
| | B2 | 31.36 ± 0.07 | — | 30.94 ± 0.08 | 28.90 ± 0.10 | 29.31 ± 0.08 |
| NRMSE | K1 | 0.140 ± 0.001 | 0.397 ± 0.004 | 0.157 ± 0.001 | 0.177 ± 0.001 | 0.170 ± 0.001 |
| | K2 | 0.134 ± 0.001 | 0.376 ± 0.004 | 0.155 ± 0.001 | 0.174 ± 0.001 | 0.150 ± 0.001 |
| | K3 | 0.112 ± 0.001 | 0.317 ± 0.003 | 0.124 ± 0.001 | 0.135 ± 0.001 | 0.116 ± 0.001 |
| | B1 | 0.158 ± 0.002 | — | 0.159 ± 0.002 | 0.201 ± 0.002 | 0.240 ± 0.003 |
| | B2 | 0.162 ± 0.002 | — | 0.171 ± 0.002 | 0.218 ± 0.003 | 0.205 ± 0.002 |

### Exemplary Results

### Exemplary Knee Studies

In the tests involving knee MR images, the exemplary systems, methods, and computer accessible medium according to the exemplary embodiments of the present disclosure can treat the highly under-sampled PDFSw k-space measurements as the “main information” and can reconstruct a PDFSw RSS image from them, using the corresponding moderately under-sampled PDw k-space measurements (which can be treated as “side information”). To evaluate the effectiveness of the exemplary systems, methods, and computer accessible medium according to the exemplary embodiments of the present disclosure in diverse settings, three tests can be performed—K1, K2, and K3—with different sampling rates in main and side information, encompassing both the non-trivial and the trivial null space cases (i.e., acceleration factors greater than and less than the number of coils), respectively. Specifically, in test K1, the null space is nontrivial, enabling approaches that exploit range-null space decomposition to be effective. In contrast, in tests K2 and K3, the null space is trivial (i.e., contains only the zero vector), in which case such approaches are expected to fail, as there is no meaningful null-space component to leverage.

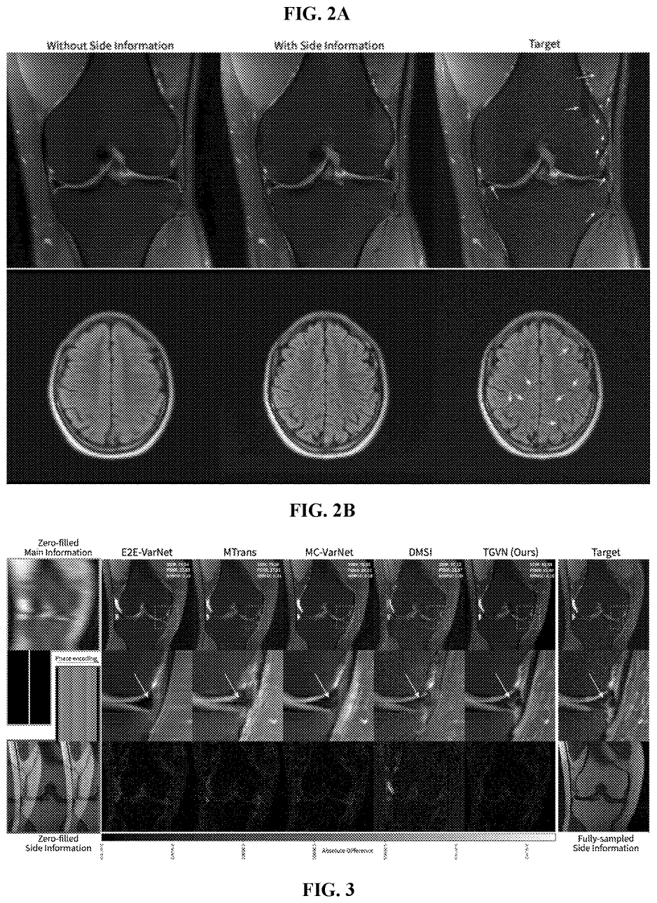

Q1: FIG. 2A shows exemplary reconstruction results for coronal PDFS images with and without using the side information. At 20×acceleration, side information according to the exemplary systems, methods, and computer accessible medium according to the exemplary embodiments of the present disclosure significantly aids reconstruction while its absence results in loss of fine anatomical details highlighted by the arrows. Table 2 further supports this finding, showing that the exemplary systems, methods, and computer accessible medium according to the exemplary embodiments of the present disclosure consistently achieve superior performance across all evaluation metrics compared to E2E-VarNet.

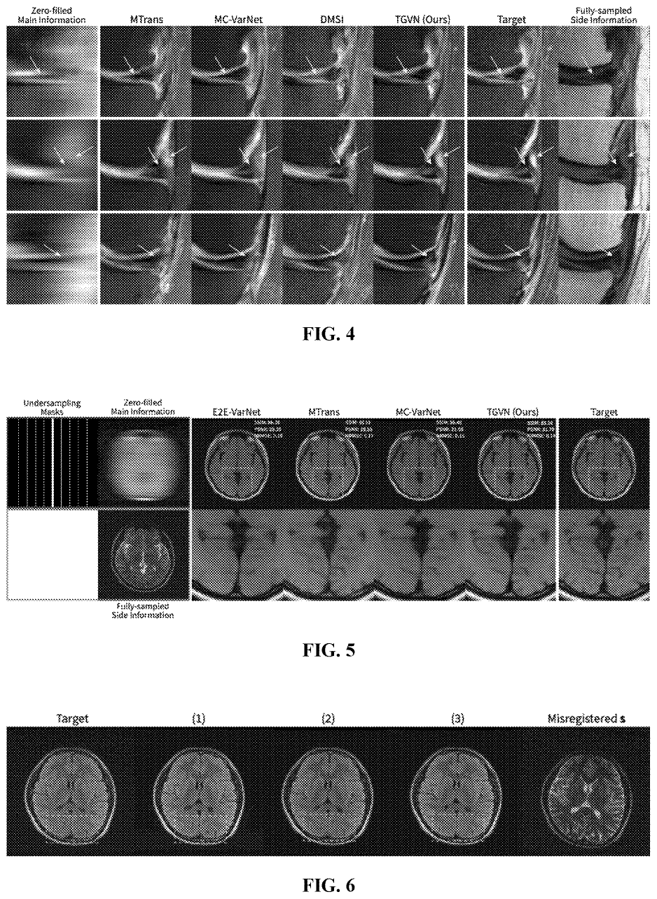

Q2: FIG. 3 compares the reconstructions of the exemplary systems, methods, and computer accessible medium according to the exemplary embodiments of the present disclosure against several baselines using side information and FIG. 4 illustrates three additional close-up cases in which the exemplary systems, methods, and computer accessible medium according to the exemplary embodiments of the present disclosure demonstrate superior recovery of clinically relevant features—namely, meniscus tears and a meniscal cyst—compared to the baselines. MTrans and MC-VarNet exhibit significant blurring of anatomical features, and DMSI suffers severely from noise amplification, which is seen clearly in the absolute difference images. The output of the exemplary systems, methods, and computer accessible medium according to the exemplary embodiments of the present disclosure is significantly superior: both overall sharpness and assorted anatomical details are better preserved in the reconstructions of the exemplary systems, methods, and computer accessible medium according to the exemplary embodiments of the present disclosure. Furthermore, the meniscus tears are distinctly more noticeable with the exemplary systems, methods, and computer accessible medium according to the exemplary embodiments of the present disclosure, highlighting that it is more effective in leveraging the side information to preserve key features in the image despite highly sparse measurements.

Notably, the meniscus tears are not well visualized in the images corresponding to the side information, which demonstrates that the exemplary systems, methods, and computer accessible medium according to the exemplary embodiments of the present disclosure are not relying excessively on features copied directly from the side information. Table 2 presents quantitative results, showing that the exemplary systems, methods, and computer accessible medium according to the exemplary embodiments of the present disclosure can achieve the best performance across all metrics with statistically significant improvements. In each test and for each evaluation metric (SSIM, PSNR, and NRMSE), a Wilcoxon signed-rank test rejected the null hypothesis at a significance level of 5, indicating a statistically significant difference, and the exemplary systems, methods, and computer accessible medium according to the exemplary embodiments of the present disclosure outperformed the next-best baseline in almost all test slices.

### Exemplary Brain Studies

In tests B1 and B2 using brain MR images, highly under-sampled FLAIR k-space and T1w k-space measurements from a single repetition is used as the “main information,” aiming to generate reconstructions with image quality approaching that of signal-averaged multi-repetition FLAIR and T1w RSS images, respectively. In both B1 and B2, the corresponding low-SNR, single-repetition T2-weighted (T2w) k-space measurements were used as “side information.” Note that the protocol includes two repetitions for FLAIR and three for T1w and T2w. Hence, using a single repetition as side information, a practical acceleration factor of 3× was achieved. To demonstrate robustness to undersampling patterns, an equispaced mask was used in these tests, providing a complementary setting to the random undersampling used in the knee tests. Fully sampled T2w images can be used as side information, in light of the low SNR and small matrix size of the acquisition. (See, e.g., Ref. 49).

Q1: FIG. 2B shows exemplary reconstruction results for axial FLAIR images with and without using the side information. At 36×practical acceleration, side information of the exemplary systems, methods, and computer accessible medium according to the exemplary embodiments of the present disclosure aids the reconstruction significantly while reconstruction without side information can result in the loss of various essential features. Moreover, Table 2 confirms that the exemplary systems, methods, and computer accessible medium according to the exemplary embodiments of the present disclosure can outperform E2E-VarNet across all evaluation metrics.

Q2: FIG. 5 juxtaposes axial T1w images reconstructed using TGVN with corresponding images reconstructed using baseline methods. Use of side information of the exemplary systems, methods, and computer accessible medium according to the exemplary embodiments of the present disclosure results in substantial improvements in image quality at the challenging practical acceleration level of 45×. The exemplary systems, methods, and computer accessible medium according to the exemplary embodiments of the present disclosure demonstrate superior performance in integrating this information compared to other methods, as evidenced by the enhanced depiction of anatomical features in the zoomed-in region and the consistently improved reconstruction metrics. Furthermore, for each quantitative evaluation metric (SSIM, PSNR, and NRMSE), a Wilcoxon signed-rank test rejected the null hypothesis at a significance level of 5, indicating that there is a statistically significant difference between sₒᵤᵣₛ and s_{base}. Table 2 reports the quantitative evaluation results for the exemplary systems, methods, and computer accessible medium according to the exemplary embodiments of the present disclosure compared to baselines. As is the case for knee validation tests, the exemplary systems, methods, and computer accessible medium according to the exemplary embodiments of the present disclosure can again achieve the best average score across all metrics. The statistically significant performance difference between the exemplary systems, methods, and computer accessible medium according to the exemplary embodiments of the present disclosure and baseline methods indicates that the side information is beneficial in guiding the reconstruction, and that the exemplary systems, methods, and computer accessible medium according to the exemplary embodiments of the present disclosure can be more effective than other methods in leveraging it.

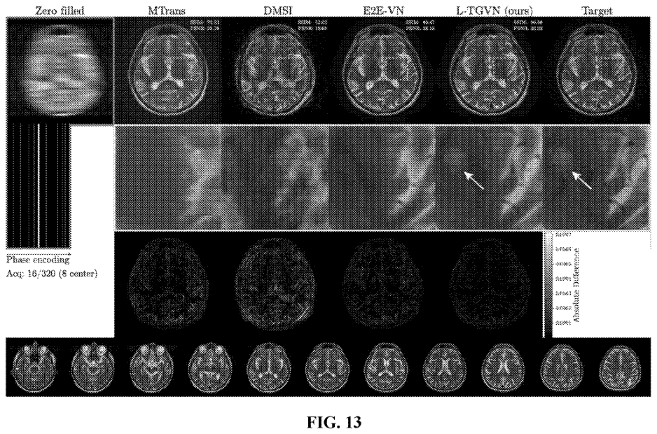

In further exemplary brain analyses directed to longitudinal side information (tests LB1 through LB3 defined above), the exemplary systems, methods, and computer accessible medium according to the exemplary embodiments of the present disclosure (in the longitudinal variant designated L-TGVN) can reconstruct axial T2w brain images from heavily undersampled k-space by leveraging one or more prior axial T2w DICOM slices from the subject's most recent prior study as side information, without requiring explicit pre-registration between prior and current scans. At a net acceleration of 20× (test LB1), reconstructions that use longitudinal priors can recover fine neuroanatomical details and pathologies that are otherwise homogenized when longitudinal priors are not used, as illustrated in FIG. 12 and FIG. 13. Quantitative evaluation at the patient level (n=81) shows that the exemplary systems, methods, and computer accessible medium according to the exemplary embodiments of the present disclosure (in the longitudinal variant designated L-TGVN) can achieve the best performance across SSIM, PSNR, and NRMSE at all three acceleration levels, with mean±standard error values of SSIM=90.89±0.36, 91.56±0.31, and 93.05±0.26(%); PSNR=31.27±0.23, 32.20±0.21, and 33.81±0.20 (dB); and NRMSE=0.253±0.006, 0.227±0.004, and 0.188±0.003 for LB1, LB2, and LB3, respectively. For comparison, the capacity-matched E2E-VarNet (which does not utilize longitudinal priors) achieves SSIM=87.65±0.39, 89.46±0.32, and 92.12±0.28(%); PSNR=29.13±0.22, 30.62±0.19, and 32.84±0.20 (dB); and NRMSE=0.322±0.006, 0.270±0.004, and 0.210±0.004 at LB1, LB2, and LB3, respectively.

Capacity-matched MTrans (see, e.g., Ref. 33) and DMSI (see, e.g., Ref. 36), adapted to longitudinal imaging by using the prior scan as conditioning input, achieve lower scores than the exemplary systems, methods, and computer accessible medium according to the exemplary embodiments of the present disclosure across all three tests (MTrans SSIM 77.05±0.49, 79.44±0.45, and 83.94±0.37; DMSI SSIM 53.78±0.44, 54.98±0.42, and 60.69±0.34 at LB1, LB2, and LB3, respectively), and tend to oversmooth the images, washing out fine structures; this failure mode is especially apparent in absolute-difference maps, which highlight widespread residual error. The exemplary systems, methods, and computer accessible medium according to the exemplary embodiments of the present disclosure can largely avoid this degradation, retaining sharper edges and better preserving subtle anatomical details (e.g., lesions), and improvements over each baseline are statistically significant by Wilcoxon signed-rank test at p<0.01 for each metric. These findings collectively confirm that longitudinal priors can noticeably enhance reconstruction quality at challenging accelerations and that the exemplary systems, methods, and computer accessible medium according to the exemplary embodiments of the present disclosure make stronger, more reliable use of prior scans than matched-capacity alternatives.

### Exemplary Ablation Studies

Exemplary ablation studies were conducted to address Q3 (whether projecting onto the ambiguous space provides benefits compared to no projection) and Q4 (how robust the exemplary systems, methods, and computer accessible medium according to the exemplary embodiments of the present disclosure is to different undersampling factors, misregistration between main and side information, and degradation in side information quality).

### Exemplary Effect of Projection

To answer Q3, the exemplary systems, methods, and computer accessible medium according to the exemplary embodiments of the present disclosure can be compared with and without the projection. In particular, the reconstruction performance of the unrolled network implementing (5) and the unrolled network implementing a modified version of (5) can be compared in which P_{δ} is replaced by the identity operator. Starting with x₀=A^{H}k, the network without the projection implements the following update equations for T iterations:

x^{t+1}=x^{t}−η^{t} [symbol] ^{H}( [symbol] x^{t}−k)−μ^{t}(x^{t}− [symbol] (s;γ^{t}))−Φ(x^{t};θ^{t}). (8)

The exemplary systems, methods, and computer accessible medium according to the exemplary embodiments of the present disclosure can apply an overall 9×undersampling mask with equispaced 15×outer undersampling and a 4 fully sampled center to the FLAIR measurements from a single repetition, achieving a practical acceleration factor of 18×. As in K1, fully sampled T2w images can be used as side information. Three Wilcoxon signed-rank tests can be performed, and they can reject the null hypotheses at a significance level of 5, concluding that there is a statistically significant difference between s_{w/} and s_{w/o}, where s_{w/} and s_{w/o} represent the SSIM, PSNR, and NRMSE scores calculated on the test dataset for the exemplary systems, methods, and computer accessible medium according to the exemplary embodiments of the present disclosure with and without the proposed projector, respectively. Furthermore, for each metric, a scatter plot comparison of s_{w/} vs s_{w/o} demonstrates that the projection can improve reconstruction quality for almost all slices in the test dataset.

### Exemplary Robustness to Degraded Side Information

Q4 can be answered by, e.g., conducting multiple tests covering misregistered, undersampled, and irrelevant side information.

Misregistration: An exemplary performance of the exemplary systems, methods, and computer accessible medium according to the exemplary embodiments of the present disclosure can be compared when the side information is perfectly registered with performance in the presence of random misregistrations simulated by small random shifts and rotations during training and/or inference. The same 9×undersampling/18×acceleration scheme can be applied as in the tests above. For each slice, three random variables, dx, dy, and dθ, can be drawn uniformly from the interval [−4,4], and side information can be translated by dx and dy pixels and rotated by dθ degrees. If the exemplary systems, methods, and computer accessible medium according to the exemplary embodiments of the present disclosure do not encounter misregistration during training, the performance can degrade substantially during inference. However, data augmentation with small random misregistrations during training can render the exemplary systems, methods, and computer accessible medium according to the exemplary embodiments of the present disclosure robust to small misregistrations during inference, as seen in FIG. 6. With such augmentation, the exemplary systems, methods, and computer accessible medium according to the exemplary embodiments of the present disclosure can still achieve significantly better scores than E2E-VarNet of the same capacity. This observation is supported by Wilcoxon tests at a 5% significance level for each metric—SSIM, PSNR, and NRMSE—demonstrating a statistically significant performance differences in favor of the exemplary systems, methods, and computer accessible medium according to the exemplary embodiments of the present disclosure with misregistered side information, as compared to E2E-VarNet, which does not utilize side information.

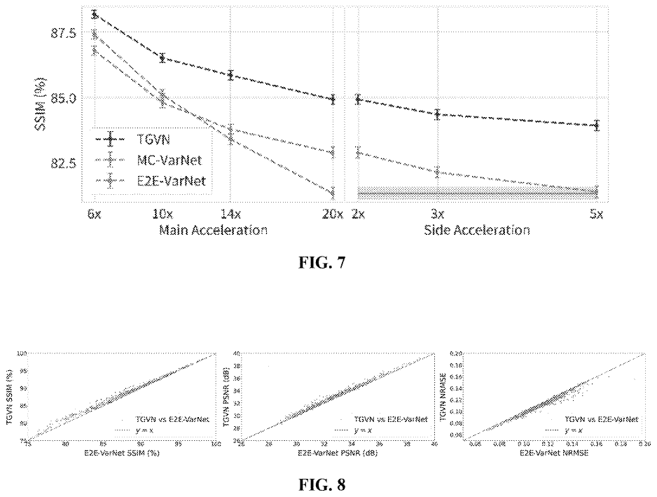

Undersampling: The performance of the exemplary systems, methods, and computer accessible medium according to the exemplary embodiments of the present disclosure and MC-VarNet can be compared at four main and three side acceleration levels, and E2E-VarNet at these four main acceleration levels using the knee data. That is, tests with all pairs of main acceleration factors (6×, 10×, 14×, and 20×random undersampling) and side acceleration factors (2×, 3×, and 5×uniform undersampling) can be performed, for both TGVN and MC-VarNet (the second-best-performing method in K1 and K2). Comparable-capacity E2E-VarNet models can also be trained at these main acceleration factors to facilitate further comparison. FIG. 7 shows the mean SSIM with standard errors as a function of the main and side acceleration factors.

The exemplary systems, methods, and computer accessible medium according to the exemplary embodiments of the present disclosure can be clearly more robust to degraded (i.e., under-sampled) side information at all acceleration factors, while MC-VarNet with 5×accelerated side information can achieve performance comparable to E2E-VarNet (which utilizes no side information). Furthermore, when varying the main acceleration, there can be a consistent performance gap between the exemplary systems, methods, and computer accessible medium according to the exemplary embodiments of the present disclosure and MC-VarNet at each acceleration level, with the exemplary systems, methods, and computer accessible medium according to the exemplary embodiments of the present disclosure performing better even at moderate main acceleration rates (e.g., 6×, 10×). The benefit of incorporating side information can become more pronounced at higher acceleration levels (e.g., 14×, 20×).

In the second part of this ablation study, the effect of fully sampled side information can be evaluated, as well as a two-stage approach where the under-sampled side information can be first reconstructed before it is utilized in reconstructing the main information. That is, three models can be compared: (I) the exemplary systems, methods, and computer accessible medium according to the exemplary embodiments of the present disclosure utilizing under-sampled side information; (II) the exemplary systems, methods, and computer accessible medium according to the exemplary embodiments of the present disclosure utilizing under-sampled side information reconstructed first with an E2E-VarNet; and (III) the exemplary systems, methods, and computer accessible medium according to the exemplary embodiments of the present disclosure utilizing fully sampled side information.

The exemplary systems, methods, and computer accessible medium according to the exemplary embodiments of the present disclosure in (I), (II), and (III) have the same number of parameters; however, inclusion of the additional high-capacity E2E-VarNet in (II) introduces a minor element of unfairness in the comparison. Specifically, the test was conducted using knee data (K2) to compare performance of the exemplary systems, methods, and computer accessible medium according to the exemplary embodiments of the present disclosure using unmodified under-sampled side information s to that using independently-reconstructed under-sampled side information or fully sampled side information \tilde{s}. Unmodified PDw under-sampled side information was provided to one version of the exemplary systems, methods, and computer accessible medium according to the exemplary embodiments of the present disclosure (I), while fully sampled PDw side information was provided to another version of the exemplary systems, methods, and computer accessible medium according to the exemplary embodiments of the present disclosure (III). For a third version of the exemplary systems, methods, and computer accessible medium according to the exemplary embodiments of the present disclosure (II), a two-stage reconstruction was used, in which the 3×under-sampled side information was first reconstructed with an E2E-VarNet (with 30 million trainable parameters, and the reconstructed result was then utilized as side information for the exemplary systems, methods, and computer accessible medium according to the exemplary embodiments of the present disclosure. As I(x; \tilde{s}|k)>I(x; s|k) for the target image x and the undersampled k-space k, one would expect (III) to outperform (I). Moreover, since x→s→ζ(s) form a Markov chain, the data processing inequality (see, e.g., Ref. 41) implies that (I) should perform at least as well as (II) in the infinite data regime.

It can be observed that the exemplary reconstruction scores improve with fully sampled side information, but the improvements can be slight and difficult to appreciate visually. Furthermore, despite having 30 million more trainable parameters to work with, the two-stage approach may not provide statistically significant improvements compared to the exemplary systems, methods, and computer accessible medium according to the exemplary embodiments of the present disclosure utilizing under-sampled side information. Quantitative evaluation results for this test are provided in Table 3. While fully sampled side information can provide the greatest benefit, moderately under-sampled side information can still be helpful, significantly improving the reconstruction compared to not having any side information (cf. Table 2, E2E-VarNet column). Furthermore, end-to-end training with under-sampled side information can perform as well as the two-stage approach-a demonstration of the data processing inequality in the finite-data regime.

Exemplary Relevance: The exemplary systems, methods, and computer accessible medium according to the exemplary embodiments of the present disclosure using the worst possible side information (e.g., complex Gaussian noise) can be compared to that of E2E-VarNet with the same refinement block capacity, and also to that of the exemplary systems, methods, and computer accessible medium according to the exemplary embodiments of the present disclosure using PDw side information. The exemplary systems, methods, and computer accessible medium according to the exemplary embodiments of the present disclosure can train and test three models with the same refinement block capacity at 20×random undersampling as in KL: (1) TGVN with 2×under-sampled PDw side information, (2) the exemplary systems, methods, and computer accessible medium according to the exemplary embodiments of the present disclosure with random complex side information (Gaussian distributed in real and imaginary channels, with zero mean and a covariance matrix matching that of the side information in (1) in order to ensure that the side information amplitude is comparable to that of the main information), and (3) E2E-VarNet.

To interpret how much the exemplary model relies on side information, each of these models can be trained using only a single cascade element. This setup can avoid ambiguity in interpreting learned parameters across multiple cascade elements, as both the trust-guidance coefficient μ and the data-consistency coefficient η are allowed to be different for each element in a multi-element cascade architecture. With a single cascade, the ratio μ/η can provide a clean proxy for the model's reliance on side information relative to the measured k-space. Since μ weights the trust guidance term and η weights the data consistency term, this ratio can reflect the model's internal assessment of the conditional mutual information I(s; x|k) during training. For the model with the best validation SSIM, the ratio μ/η in (1) is 0.918, while for random side information in (2) it is 0.032, clearly indicating that the exemplary systems, methods, and computer accessible medium according to the exemplary embodiments of the present disclosure can learn to ignore side information when it is irrelevant.

Exemplary quantitative evaluation results are provided in Table 4, demonstrating that there is no statistically significant difference between the exemplary systems, methods, and computer accessible medium according to the exemplary embodiments of the present disclosure with irrelevant side information (2) and an E2E-VarNet (3), provided that the irrelevant side information—without any distribution shift—is present during both training and inference.

TABLE 3
Quantitative evaluation results for the
second part of the Ablation Study.

| Metric/Model | (I) | (II) | (III) |
|---|---|---|---|
| SSIM (%) | 85.52 ± 0.19 | 85.56 ± 0.19 | 85.97 ± 0.18 |
| PSNR (dB) | 31.31 ± 0.07 | 31.41 ± 0.07 | 31.60 ± 0.07 |
| NRMSE | 0.13 ± 0.001 | 0.13 ± 0.001 | 0.13 ± 0.001 |

TABLE 4
Exemplary quantitative evaluation results
for the Ablation Study Relevance.

| Metric/Model | (1) | (2) | (3) |
|---|---|---|---|
| SSIM (%) | 83.75 ± 0.20 | 80.15 ± 0.24 | 80.19 ± 0.24 |
| PSNR (dB) | 30.03 ± 0.07 | 28.47 ± 0.07 | 28.47 ± 0.07 |
| NRMSE | 0.16 ± 0.001 | 0.19 ± 0.001 | 0.19 ± 0.001 |

### Exemplary Conclusion

The exemplary systems, methods, and computer accessible medium according to the exemplary embodiments of the present disclosure provide a novel framework that demonstrates the power of leveraging side information in solving LIPs, with specific application to the MR image reconstruction problem. By learning to eliminate solutions from the ambiguous space of the forward operator while remaining faithful to acquired measurements through data consistency, the principled approach of the exemplary systems, methods, and computer accessible medium according to the exemplary embodiments of the present disclosure can make maximal use of relevant side information while minimizing the risk of hallucinations. Of primary importance is that, when incorporated effectively by the exemplary systems, methods, and computer accessible medium according to the exemplary embodiments of the present disclosure, subject-specific side information can significantly improve reconstruction quality and preserve anatomical and pathological features while avoiding hallucinations, even at undersampling levels as high as e.g., 20×, and even with moderately-under-sampled or low-quality side information. High levels of acceleration according to the exemplary systems, methods, and computer accessible medium according to the exemplary embodiments of the present disclosure, meanwhile, can have a transformative impact in healthcare, by improving imaging efficiency in traditional settings, and also by enabling use of lower-quality data from accessible imaging devices for widespread health monitoring at the population level.

In longitudinal settings, the exemplary systems, methods, and computer accessible medium according to the exemplary embodiments of the present disclosure can further demonstrate that a patient's most recent prior scan can serve as highly informative, subject-specific side information for reconstructing a follow-up scan from heavily undersampled measurements, provided that the influence of the prior is constrained to remain data-consistent by the trust mechanism. This exemplary trust-guided design helps preserve fine anatomical detail and clinically relevant interval changes (e.g., pathology progression) while reducing the risk of over-reliance on outdated information in the presence of temporal change, inter-scan misalignment, and protocol drift, and does so without requiring explicit pre-registration between visits. The longitudinal variant can be extended in further exemplary embodiments to leverage multiple prior visits and multiple prior series (e.g., T2-weighted and FLAIR), to incorporate richer non-image longitudinal context such as radiology reports and clinical metadata, and to provide uncertainty-aware operation that flags regions where prior information conflicts with current measurements, thereby enabling adaptive acquisition and reconstruction.

FIG. 11 shows an exemplary overview of longitudinal trust-guided disambiguation of solutions to the LIP according to an exemplary embodiment of the present disclosure. In FIG. 11, a longitudinal prior 1100, comprising a plurality of axial T2-weighted slices from a most recent prior study of a subject, can be used as side information to reconstruct a target axial T2-weighted slice in a follow-up exam from highly undersampled k-space measurements. The longitudinal prior 1100 can be depicted as a vertical stack of axial brain MRI images representing multiple slices from the same series in the latest prior study, which can serve as subject-specific side information for the reconstruction process. An L-TGVN processing block 1105 can receive an initial estimate input 140 derived from the undersampled measurements and can process this input along with the longitudinal prior 1100. Within the L-TGVN processing block 1105, a trusted space 120 can correspond to large singular values of the forward operator 110, representing information that can be reliably determined from the acquired measurements, while an ambiguous space 125 can correspond to small singular values of the forward operator 110, representing information that cannot be reliably distinguished based on the measured data alone. The output of the L-TGVN processing block 1105 can be a final reconstructed image 1110 that preserves fine neuroanatomical details and clinically relevant features despite the high level of undersampling in the current-session k-space measurements. Eliminated ambiguous space components 1115, representing solutions from the ambiguous space 125 that are inconsistent with the side information, can be removed through the trust-guided mechanism while preserving data consistency with the acquired measurements.

FIG. 12 shows exemplary axial T2-weighted reconstructions from a test split for a net acceleration of 20×. As shown in FIG. 12, using longitudinal priors can significantly enhance reconstruction quality. The exemplary systems, methods, and computer accessible medium according to the exemplary embodiments of the present disclosure (L-TGVN) can recover fine neuroanatomical details and pathologies (indicated by arrows), matching the target, whereas without longitudinal priors, key brain structures can be homogenized and highlighted pathologies can become difficult to discern.

FIG. 13 shows exemplary axial T2-weighted brain reconstructions at a net acceleration of 20×, demonstrating the effectiveness of the exemplary systems, methods, and computer accessible medium according to the exemplary embodiments of the present disclosure (L-TGVN) in utilizing longitudinal priors. FIG. 13 can include full field-of-view images comparing different reconstruction methods, including zero-filled, MTrans, DMSI, E2E-VarNet, and the exemplary systems, methods, and computer accessible medium according to the exemplary embodiments of the present disclosure (L-TGVN), along with a target reference. Zoomed-in regions can highlight areas where a lesion is clearly visible only in the reconstruction of the exemplary systems, methods, and computer accessible medium according to the exemplary embodiments of the present disclosure among the methods utilizing side information. Absolute difference maps can show reconstruction errors relative to the target. Side-information slices acquired in a prior imaging session can exhibit rigid misalignment relative to the current scan due to different slice prescription and patient positioning between the prior and current imaging sessions.

A number of ablation studies were performed in order to assess both the value of trust guidance and the residual risk of hallucinations of the exemplary systems, methods, and computer accessible medium according to the exemplary embodiments of the present disclosure when side information is degraded in quality or poorly matched to the main information. The ablation results demonstrate that projection onto the ambiguous space can indeed provide notable performance benefits. These results also show that on average, as long as training is adapted and there is no severe distribution shift, side information cannot hurt the reconstruction of the exemplary systems, methods, and computer accessible medium according to the exemplary embodiments of the present disclosure. In the worst case-when s and x are conditionally independent given k—the exemplary systems, methods, and computer accessible medium according to the exemplary embodiments of the present disclosure can learn to ignore the side information by shrinking the trust-guidance coefficient μ^{t}.

### Exemplary Training and Evaluation Details

The exemplary systems, methods, and computer accessible medium according to the exemplary embodiments of the present disclosure can use the MS-SSIM-L1 (see, e.g., Refs. 50, 51) loss function and can employ the ADAM optimizer (see, e.g., Ref. 52), with batch size of e.g., one per GPU and with default parameters including a uniform kernel of size e.g., 33×33 and k-values of 0.01 and 0.03, across 5 values of a (0.5, 1.0, 2.0, 4.0, 8.0), in both training and validation phases. The loss can be calculated between the reconstructed and the ground-truth root-sum-of-squares (RSS) (see, e.g., Ref. 53) images. A starting learning rate of e.g., 3×10⁻⁴ can be used with exponential decay with the decay parameter 0.98. These parameters can be determined through a grid search on the validation set. The training of the exemplary systems, methods, and computer accessible medium according to the exemplary embodiments of the present disclosure can span e.g., 100 epochs, with the best model parameters selected based on the validation loss. All models can be trained and tested on e.g., 4×NVIDIA A100 GPUs using PyTorch for 10 days in knee tests and 2 days in brain tests. Sensitivity maps can be estimated with a separate module (SME), as in. (See, e.g., Ref. 6). The ASC (H), refinement (Φ), and SME modules can all be implemented using a U-Net model, as illustrated in FIG. 1E, which can consist of e.g., four down-sampling and up-sampling paths, complemented by skip connections 170. Each down-sampling step can employ an average pooling layer 175, and each up-sampling step can employ a transposed convolution layer 180. Each path can be equipped with e.g., two 3×3 convolutions, followed by instance normalization and leaky ReLU activation 185. (See, e.g., Refs. 54 and 55)

The first convolution layer can output e.g., 21 channels in the ASC and refinement networks and 8 in the SME network, with the number doubling in each subsequent layer, as in (see, e.g., Ref. 6). In the brain tests, the exemplary systems, methods, and computer accessible medium according to the exemplary embodiments of the present disclosure can comprise T=10 TGVN blocks having approximately 67.3 million trainable parameters in total. In the knee tests, it includes T=14 TGVN blocks, resulting in about 94 million trainable parameters. For enhanced numerical stability in training, complex-valued layer normalization and its inverse operator can be used, similar to (see, e.g., Ref. 6). For example, each network pass, denoted as x [symbol] Φ(x), can be executed as x [symbol] T⁻¹(Φ(Tx)). For an input tensor of shape (B, 2, H, W), where B is the batch size, the two channels can correspond to the real and imaginary components of the image. The normalization process T can adjust each sample so that the mean of each channel across the spatial dimensions (H, W) is zero, and the standard deviation can be set to one. Furthermore, T ensures that the real and imaginary channels are decorrelated, resulting in zero covariance between them. This can be achieved by computing the 2×2 covariance matrix of the two channels and performing a linear combination with the transpose of the Cholesky decomposition of the inverse of the covariance matrix.

This exemplary normalization step can facilitate the network to handle the real and imaginary parts without any inherent bias or unintended correlation, ultimately improving the stability and performance of the exemplary systems, methods, and computer accessible medium according to the exemplary embodiments of the present disclosure. In both knee and brain tests, the approximate projector as described above was implemented for the trust-guidance term with 10 conjugate gradient iterations, and the threshold parameter δ was learned during training. Use of the approximate projector can circumvent the need for a computationally expensive SVD operation. For example, in K3, the forward operator can have more than 200,000 rows and 200,000 columns, making explicit SVD calculation prohibitively costly.

For the exemplary longitudinal variant (L-TGVN), the trust-guidance module H can be implemented as a dual-encoder, shared-decoder U-Net as described above, in which the prior-encoder processes a small stack (e.g., Nₛ=11) of neighboring slices from the most relevant prior series selected based on metadata (e.g., z-axis position) and the current-iterate encoder processes the complex-valued current estimate mapped to two real channels; at each resolution, features from the two encoders can be fused (e.g., concatenation followed by a 1×1 convolution) to form shared skip connections for a single decoder that outputs a complex-valued proposal with the same spatial support as the current reconstruction. Conditioning the exemplary trust-guidance module on the current iterate can allow the network to learn implicit alignment and protocol adaptation, thereby avoiding a separate pre-registration pipeline and accommodating protocol drift and scanner differences between the prior and current sessions. For a fair comparison with baseline methods that also leverage longitudinal priors, the exemplary systems, methods, and computer accessible medium according to the exemplary embodiments of the present disclosure (L-TGVN) and the baselines can be configured to have a similar number of trainable parameters (e.g., approximately 500 million).

The exemplary systems, methods, and computer accessible medium according to the exemplary embodiments of the present disclosure (L-TGVN), MTrans, and E2E-VarNet can be trained on, e.g., 8×NVIDIA A100 GPUs with 80 GB VRAM with an equivalent batch size of 64 for 20,000 gradient steps, with parameters saved at each epoch and the model having the best validation SSIM selected for testing. DMSI can be trained for, e.g., 70,000 gradient steps with the same setup (as it requires longer training), with the model having the best validation EDM loss (see, e.g., Ref. 63) selected for testing. Training can increase or maximize a chosen similarity metric (e.g., SSIM) between the final iterate x^{T} and a ground-truth target image x*. In still further exemplary embodiments, the prior input can comprise multiple series from a given prior study (e.g., T2-weighted and FLAIR), or images from multiple prior imaging sessions, and the dual-encoder structure can be extended to process each additional source in parallel with feature-level fusion. Additional training, preprocessing, and data-selection details are provided in the accompanying repository.

### Exemplary Baselines

The officially released repositories can be used for the baselines instead of re-implementing the models. As a result, there was only a need to adjust the model capacities to match that of the exemplary systems, methods, and computer accessible medium according to the exemplary embodiments of the present disclosure in each setting and modify the training learning rates to adapt to the fastMRI and M4Raw datasets.

E2E-VarNet: For example, the MS-SSIM-L1 loss function can be used and the ADAM optimizer can be employed, with batch size of e.g., one per GPU and with the same hyperparameters as used in the exemplary systems, methods, and computer accessible medium according to the exemplary embodiments of the present disclosure. The loss can be calculated between the reconstructed and the ground-truth root-sum-of-squares (RSS) images. A starting learning rate of, e.g., 3×10⁻⁴ can be used with exponential decay with parameters 0.98. These parameters can be determined through a grid search on the validation set. The training can span e.g., 100 epochs, with the best model parameters selected based on the validation loss. All the models can be trained and tested on e.g., 4×NVIDIA A100 GPUs using PyTorch for 3 days in knee tests and 1 day in brain tests. The refinement and SME modules can share the same U-Net architecture as the exemplary systems, methods, and computer accessible medium according to the exemplary embodiments of the present disclosure. The first convolution layer can output e.g., 30 channels in the refinement network and 8 in the SME network, with the number doubling in each subsequent layer, as in (see, e.g., Ref. 6). In the brain tests, it can comprise e.g., T=10 VarNet blocks having approximately 68.7 million trainable parameters in total. In the knee tests, it can include e.g., T=14 VarNet blocks, resulting in about 96 million trainable parameters.

MTrans: An exemplary CrossCMMT model can be used and the hidden dimension can be set to 17 and 14 for the knee and brain tests, respectively. Input sizes can be chosen as the image matrix size, without any resolution change. Parameters P1, P2 can be set to e.g., 8, and CTDEPTH and TRANSFORMER_NUM_HEADS can be set to e.g., 4, and TRANSFORMER_MLP_RATIO can be set to e.g., 3. With these parameters, the knee model can have e.g., 98.3 million and the brain model e.g., 66.1 million trainable parameters. Initial learning rate can be set to e.g., 2×10⁻⁴. One model can be trained without scheduling and one model can be scheduled with an exponential decay e.g., γ=0.99. Both models can be trained for e.g., 100 epochs on 4×NVIDIA A100 GPUs using PyTorch with a unit batch size per GPU. The best-performing model, according to the average validation SSIM, was the one trained with exponential decay. Training can span approximately 4 days for knee and 1 day for brain.

MC-VarNet: Exemplary in_channel and channel_fea parameters can be set to 264 and 224 respectively without changing the default iter_num (4), which can result in e.g., 94.1 and 67.8 million trainable parameters for the knee and brain tests, respectively. For the brain tests, an initial learning rate of e.g., 10⁻⁴ can be used, and one model without scheduling and one model scheduled with an exponential decay parameter 0.99. can be trained For the knee tests, learning rates on the order of e.g., 10⁻⁴ can result in unstable training, so an initial learning rate of 10⁻⁵ can be chosen and can again train one model without scheduling and one with exponential decay parameter 0.99. Each model can be trained for e.g., 100 epochs on 4×NVIDIA A100 GPUs using PyTorch with a unit batch size per GPU. The best-performing model, according to the average validation SSIM, was the one trained with exponential decay. Training can span approximately 7 days for knee and 1 day for brain.

### Exemplary Evaluation

For the exemplary SSIM metric, an e.g., 7×7 uniform kernel can be utilized, along with the standard k-values of 0.01 and 0.03. The range parameter can be given as input to the SSIM calculation and can be set to the maximum pixel value of the corresponding volume. In Wilcoxon signed rank tests, D can be the distribution of pairwise differences sₒᵤᵣₛ−s_{base}. Then under the alternate hypothesis, D can be “stochastically greater than a distribution symmetric about zero” for SSIM and PSNR and “stochastically less than a distribution symmetric about zero” for NRMSE. The average inference time on e.g., an NVIDIA A100 GPU for the exemplary systems, methods, and computer accessible medium according to the exemplary embodiments of the present disclosure can be measured to be e.g., 468 ms per slice. In comparison, the reconstruction times per slice can be e.g., 132 ms for E2E-VarNet, 43 ms for MTrans, 495 ms for MC-VarNet, and 199,206 ms for DMSI—more than—slower than the second-slowest method, MC-VarNet.

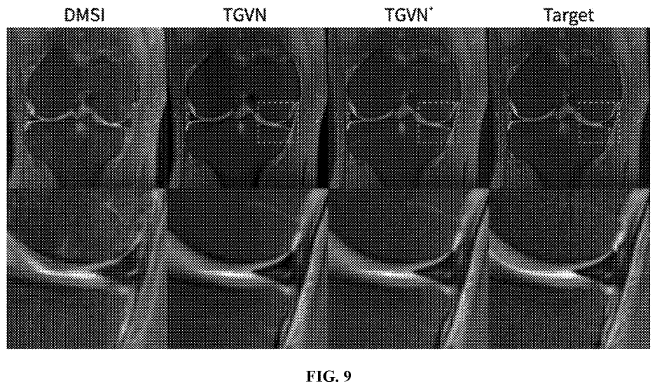

Exemplary scatter plots comparing SSIM, PSNR, and NRMSE scores on the test split for the exemplary systems, methods, and computer accessible medium according to the exemplary embodiments of the present disclosure and the second-best method for K1-3, B1-2 can demonstrate that the exemplary systems, methods, and computer accessible medium according to the exemplary embodiments of the present disclosure can consistently improve reconstruction quality for almost all slices in the test dataset. One example is provided in FIG. 8. To enhance visual comparison despite the inherent denoising properties of the exemplary systems, methods, and computer accessible medium according to the exemplary embodiments of the present disclosure, FIG. 9 shows additional reconstructions with noise added deliberately to match the subjective noise level of the target images, as previously implemented in. (See, e.g., Ref. 56). This can preserve not only anatomical and pathological features but also overall image appearance as compared with target images.

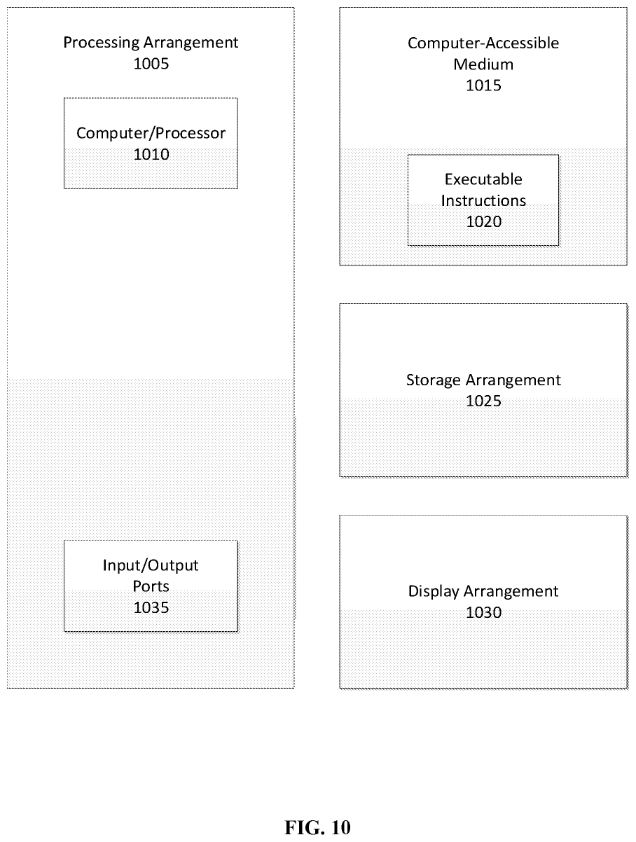

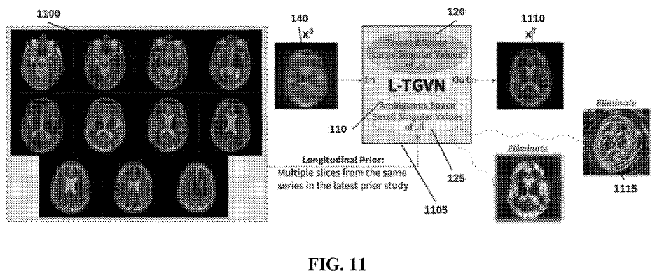

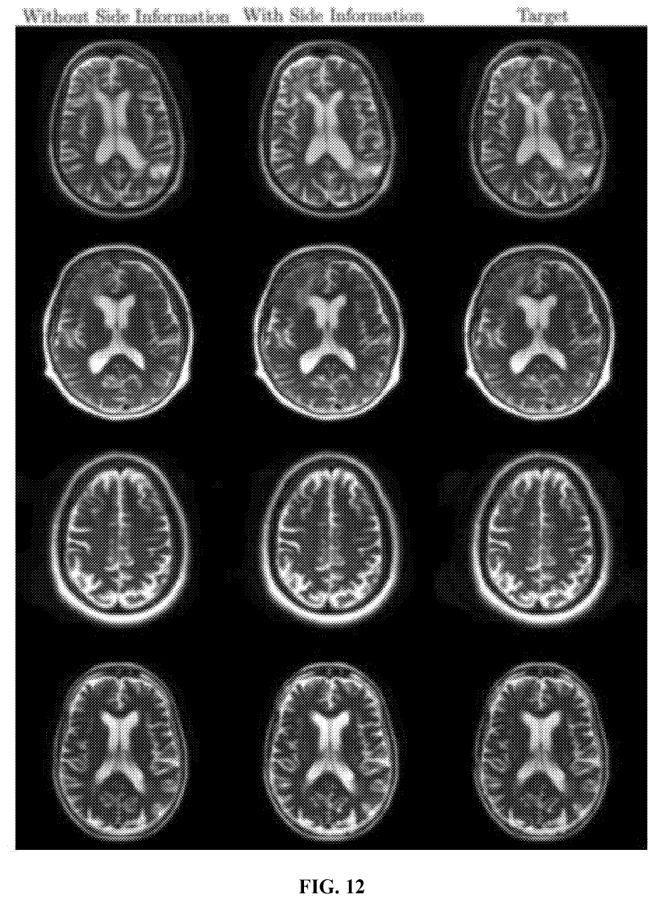

FIG. 10 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1005. Such processing/computing arrangement 1005 can be, for example entirely or a part of, or include, but not limited to, a computer/processor 1010 that can include, for example one or more microprocessors, and use instructions stored on a computer-accessible medium (e.g., RAM, ROM, hard drive, or other storage device).

As shown in FIG. 10, for example a computer-accessible medium 1015 (e.g., as described herein above, a storage device such as a hard disk, floppy disk, memory stick, CD-ROM, RAM, ROM, etc., or a collection thereof) can be provided (e.g., in communication with the processing arrangement 1005). The computer-accessible medium 1015 can contain executable instructions 1020 thereon. In addition, or alternatively, a storage arrangement 1025 can be provided separately from the computer-accessible medium 1015, which can provide the instructions to the processing arrangement 1005 so as to configure the processing arrangement to execute certain exemplary procedures, processes, and methods, as described herein above, for example. Further, the exemplary processing arrangement 1005 can be provided with or include an input/output ports 1035, which can include, for example a wired network, a wireless network, the internet, an intranet, a data collection probe, a sensor, etc. As shown in FIG. 10, the exemplary processing arrangement 1005 can be in communication with an exemplary display arrangement 1030, which, according to certain exemplary embodiments of the present disclosure, can be a touch-screen configured for inputting information to the processing arrangement in addition to outputting information from the processing arrangement, for example. Further, the exemplary display arrangement 1030 and/or a storage arrangement 1025 can be used to display and/or store data in a user-accessible format and/or user-readable format.

In some exemplary embodiments, the projecting of a quantity derived from the individualized side information onto the ambiguous space can comprise applying an approximate projector computed via a predetermined number of conjugate gradient iterations, thereby avoiding explicit singular value decomposition of the forward operator. This exemplary approach can reduce computational cost while maintaining accuracy in the trust-guidance projection.

In some exemplary embodiments, the solution of the inverse problem can be computed via an optimization problem, and the ambiguity can be resolved through incorporating an ambiguous space consistency regularization term into the optimization problem. The exemplary systems, methods, and computer accessible medium according to the exemplary embodiments of the present disclosure can further comprise generating a learning model trained to resolve at least one ambiguity. The learning model can comprise a conditional guidance network whose output depends jointly on the individualized side information and on the current iterate of the solution of the inverse problem. The conditional guidance network can comprise a first encoder processing the individualized side information, a second encoder processing the current iterate, and a shared decoder receiving fused features from the first encoder and the second encoder, thereby learning implicit alignment without a separate pre-registration pipeline. The learning model can be trained end-to-end in a supervised manner by maximizing a similarity metric, wherein the similarity metric can comprise a structural similarity index, between a final iterate of the reconstruction and a ground-truth target image.

In some exemplary embodiments, the ambiguity can arise from a system of equations governing the inverse problem being underdetermined. The individualized side information can comprise one or more images of a different contrast from a same imaging session as a current image, wherein the current image corresponds to the solution of the inverse problem. The individualized side information can also comprise one or more prior images acquired in a different imaging session from a same subject, wherein the imaging can be performed with a same or different scanner. The different scanner can comprise a scanner of a different vendor, a different model, a different field strength, a different coil configuration, or a different software version. The prior image(s) and the current image can differ in one or more acquisition parameters. A plurality of prior images can be used as the individualized side information for reconstruction of a given current image. The individualized side information can further comprise one or more images acquired using a different imaging modality from a current image.

In some exemplary embodiments, the exemplary systems, methods, and computer accessible medium according to the exemplary embodiments of the present disclosure can enable scanners with memory allowing personalized and accelerated image acquisition when individualized side information is available from a particular individual. The individualized side information can be incorporated into reconstruction of a current image with a trust guidance mechanism that prevents hallucination or overwriting of data from the accelerated acquisition. Incorporating the individualized side information can comprise applying trust guidance that constrains the influence of the individualized side information to be consistent with measurement data acquired in the accelerated acquisition. The accelerated acquisition can enable higher acceleration factors than would otherwise be feasible. The higher acceleration can be achieved by undersampling of measurement data, or by reduction of averaging or repetition of acquisitions.

In some exemplary embodiments, the exemplary systems, methods, and computer accessible medium according to the exemplary embodiments of the present disclosure can improve image quality using individualized side information, thereby enabling use of image acquisition hardware with reduced cost or increased accessibility. The individualized side information can be incorporated into reconstruction of a current image with a trust guidance mechanism that prevents hallucination or overwriting of measurement data. Incorporating the individualized side information can comprise applying trust guidance that constrains the influence of the individualized side information to be consistent with measurement data. The individualized side information can enable higher signal-to-noise ratio and contrast-to-noise ratio than would otherwise be feasible.

According to the exemplary embodiments of the present disclosure, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the appended claims, and may include other examples that occur to those skilled in the art. Such other examples are intended to be within the scope of the appended claims if they have structural elements that do not differ from the literal language of the appended claims, or if they include equivalent structural elements with insubstantial differences from the literal language of the appended claims.

### EXEMPLARY REFERENCES

The following references are hereby incorporated by reference, in their entireties:
[1] O. Ronneberger, P. Fischer, and T. Brox, “U-Net: Convolutional Networks for Biomedical Image Segmentation,” in Proc. Int. Conf. Med. Image comput. Comput.-assist. intervent., 2015, pp. 234-241.
[2] M. Lustig, D. L. Donoho, and J. M. Pauly, “Sparse MRI: The application of compressed sensing for rapid MR imaging,” Magn. Reson. Med., vol. 58, no. 6, pp. 1182-1195, 2007.
[3] S. Ravishankar and Y. Bresler, “MR Image Reconstruction From Highly Undersampled k-Space Data by Dictionary Learning,” IEEE Trans. Med. Imaging, vol. 30, no. 5, pp. 1028-1041, 2010.
[4] J. Caballero, A. N. Price, D. Rueckert, and J. V. Hajnal, “Dictionary Learning and Time Sparsity for Dynamic MR Data Reconstruction,” IEEE Trans. Med. Imaging, vol. 33, no. 4, pp. 979-994, 2014.
[5] K. Hammernik et al., “Learning a variational network for reconstruction of accelerated MRI data,” Magn. Reson. Med., vol. 79, no. 6, pp. 3055-3071, 2018.
[6] A. Sriram et al., “End-to-End Variational Networks for Accelerated MRI Reconstruction,” in Proc. Int. Conf. Med. Image comput. Comput.-assist. intervent., 2020, pp. 64-73.
[7] H. K. Aggarwal, M. P. Mani, and M. Jacob, “MoDL: Model-Based Deep Learning Architecture for Inverse Problems,” IEEE Trans. Med. Imaging, vol. 38, no. 2, pp. 394-405, 2019.
[8] J. Xiang, Y. Dong, and Y. Yang, “FISTA-Net: Learning a Fast Iterative Shrinkage Thresholding Network for Inverse Problems in Imaging,” IEEE Trans. Med Imaging, vol. 40, no. 5, pp. 1329-1339, 2021.
[9] A. Bora, A. Jalal, E. Price, and A. G. Dimakis, “Compressed Sensing Using Generative Models,” in Proc. Int. Conf. Mach. learn., 2017, pp. 537-546.
[10] H. Chung and J. C. Ye, “Score-based diffusion models for accelerated MRI,” Med Image Anal., vol. 80, p. 102479, 2022.
[11] Y. Song, L. Shen, L. Xing, and S. Ermon, “Solving Inverse Problems in Medical Imaging with Score-Based Generative Models,” in Proc. Int. Conf. Learn. represent., 2022.
[12] A. Radmanesh et al., “Exploring the Acceleration Limits of Deep Learning Variational Network-based Two-dimensional Brain MRI,” Radiol. Artif. Intell., vol. 4, no. 6, p. e210313, 2022.
[13] L. Weizman, Y. C. Eldar, and D. Ben Bashat, “Compressed sensing for longitudinal MRI: An adaptive-weighted approach,” Med. Phys., vol. 42, no. 9, pp. 5195-5208, 2015.
[14] L. Weizman, Y. C. Eldar, and D. Ben Bashat, “Reference-based MRI,” Med. Phys., vol. 43, no. 10, pp. 5357-5369, 2016.
[15] M. J. Ehrhardt and M. M. Betcke, “Multicontrast MRI Reconstruction with Structure-Guided Total Variation,” SIAM J. Imag. Sci., vol. 9, no. 3, pp. 1084-1106, 2016.
[16] P. Song, L. Weizman, J. F. C. Mota, Y. C. Eldar, and M. R. D. Rodrigues, “Coupled Dictionary Learning for Multi-Contrast MRI Reconstruction,” IEEE Trans. Med. Imaging, vol. 39, no. 3, pp. 621-633, 2020.
[17] B. Zhou and S. K. Zhou, “DuDoRNet: Learning a Dual-Domain Recurrent Network for Fast MRI Reconstruction with Deep T1 Prior,” in Proc. IEEE/CVF conf Comput. Vis. Pattern recognit., 2020, pp. 4273-4282.
[18] P. Lei, F. Fang, G. Zhang, and T. Zeng, “Decomposition-Based Variational Network for Multi-Contrast MRI Super-Resolution and Reconstruction,” in Proc. IEEE int. Conf Comput. vis., 2023, pp. 21296-21306.
[19] P. C. Hansen, Discrete Inverse Problems: Insight and Algorithms. SIAM, 2010.
[20] L. Landweber, “An Iteration Formula for Fredholm Integral Equations of the First Kind,” Amer. J. Math., vol. 73, no. 3, pp. 615-624, 1951.
[21] J. A. Fessler, N. H. Clinthorne, and W. L. Rogers, “Regularized emission image reconstruction using imperfect side information,” IEEE Trans. Nucl. Sci., vol. 39, no. 5, pp. 1464-1471, 1992.
[22] G. Gindi, M. Lee, A. Rangarajan, and I. G. Zubal, “Bayesian reconstruction of functional images using anatomical information as priors,” IEEE Trans. Med. Imaging, vol. 12, no. 4, pp. 670-680, 1993.
[23] J. P. Haldar, D. Hernando, S.-K. Song, and Z.-P. Liang, “Anatomically constrained reconstruction from noisy data,” Magn. Reson. Med., vol. 59, no. 4, pp. 810-818, 2008.
[24] B. Wu, R. P. Millane, R. Watts, and P. J. Bones, “Prior estimate-based compressed sensing in parallel MRI,” Magn. Reson. Med., vol. 65, no. 1, pp. 83-95, 2011.
[25] X. Peng, H.-Q. Du, F. Lam, S. D. Babacan, and Z.-P. Liang, “Reference-driven MR image reconstruction with sparsity and support constraints,” in Proc. IEEE int. Symp. Biomed. imaging, 2011, pp. 89-92.
[26] B. Bilgic, V. K. Goyal, and E. Adalsteinsson, “Multi-contrast reconstruction with Bayesian compressed sensing,” Magn. Reson. Med., vol. 66, no. 6, pp. 1601-1615, 2011.
[27] H. Du and F. Lam, “Compressed sensing MR image reconstruction using a motion-compensated reference,” Magn. Reson. Imaging, vol. 30, no. 7, pp. 954-963, 2012.
[28] J. Huang, C. Chen, and L. Axel, “Fast multi-contrast MRI reconstruction,” Magn. Reson. Imag., vol. 32, no. 10, pp. 1344-1352, 2014.
[29] G. Li et al., “Incorporation of image data from a previous examination in 3D serial MR imaging,” Magn. Reson. Mater. Phys. Biol. Med., vol. 28, pp. 413-425, 2015.
[30] P. Lei, F. Fang, G. Zhang, and M. Xu, “Deep Unfolding Convolutional Dictionary Model for Multi-Contrast MRI Super-resolution and Reconstruction,” in Proc. Int. Joint conf. Artif. intell., 2023, pp. 1008-1016.
[31] L. Xiang et al., “Ultra-fast T2-weighted MR reconstruction using complementary T1-weighted information,” in Proc. Int. Conf. Med. Image comput. Comput.-assist. intervent., Springer, 2018, pp. 215-223.
[32] L. Xiang et al., “Deep-Learning-Based Multi-Modal Fusion for Fast MR Reconstruction,” IEEE Trans. Biomed. Eng., vol. 66, no. 7, pp. 2105-2114, 2019.
[33] C.-M. Feng et al., “Multimodal Transformer for Accelerated MR Imaging,” IEEE Trans. Med. Imaging, vol. 42, no. 10, pp. 2804-2816, 2022.
[34] S. U. H. Dar, M. Yurt, M. Shahdloo, M. E. Ildiz, B. Tinaz, and T. Çukur, “Prior-Guided Image Reconstruction for Accelerated Multi-Contrast MRI via Generative Adversarial Networks,” IEEE J. Sel. Topics Signal Process., vol. 14, no. 6, pp. 1072-1087, 2020.
[35] V. A. Kelkar and M. Anastasio, “Prior Image-Constrained Reconstruction using Style-Based Generative Models,” in Proc. Int. Conf. Mach. learn., 2021, pp. 5367-5377.
[36] B. Levac, A. Jalal, K. Ramchandran, and J. I. Tamir, “MRI Reconstruction with Side Information using Diffusion Models,” in Proc. Asilomar conf. Signals, syst., comput., 2023, pp. 1436-1442.
[37] J. Schwab, S. Antholzer, and M. Haltmeier, “Deep null space learning for inverse problems: Convergence analysis and rates,” Inverse Problems, vol. 35, no. 2, p. 025008, 2019.
[38] Y. Wang, Y. Hu, J. Yu, and J. Zhang, “GAN Prior based Null-Space Learning for Consistent Super-Resolution,” in Proc. AAAI conf Artif. intell., 2023, pp. 2724-2732.
[39] Y. Wang, J. Yu, and J. Zhang, “Zero-Shot Image Restoration Using Denoising Diffusion Null-Space Model,” in Proc. Int. Conf. Learn. represent., 2023.
[40] J. Chen, J. Jiang, F. Wu, and J. Zheng, “Null Space Matters: Range-Null Decomposition for Consistent Multi-Contrast MRI Reconstruction,” in Proc. AAAI conf. Artif. intell., 2024, pp. 1081-1090.
[41] T. M. Cover and J. A. Thomas, Elements of Information Theory, 2nd ed. Wiley-Interscience, 2006.
[42] M. J. Muckley et al., “Results of the 2020 fastMRI challenge for machine learning MR image reconstruction,” IEEE Trans. Med. Imaging, vol. 40, no. 9, pp. 2306-2317, 2021.
[43] K. P. Pruessmann, M. Weiger, M. B. Scheidegger, and P. Boesiger, “SENSE: Sensitivity encoding for fast MRI,” Magn. Reson. Med., vol. 42, no. 5, pp. 952-962, 1999.
[44] M. R. Hestenes, E. Stiefel, et al., “Methods of Conjugate Gradients for Solving Linear Systems,” J. Res. Natl. Bur. Stand., vol. 49, no. 6, pp. 409-436, 1952.
[45] Z. Wang, A. C. Bovik, H. R. Sheikh, and E. P. Simoncelli, “Image quality assessment: From error visibility to structural similarity,” IEEE Trans. Image Process., vol. 13, no. 4, pp. 600-612, 2004.
[46] F. Wilcoxon, “Individual Comparisons by Ranking Methods,” Biomed. Bull., vol. 1, no. 6, pp. 80-83, 1945.
[47] F. Knoll et al., “FastMRI: A Publicly Available Raw k-Space and DICOM Dataset of Knee Images for Accelerated MR Image Reconstruction Using Machine Learning,” Radiol. Artif. Intell., vol. 2, no. 1, p. e190007, 2020.
[48] R. Zhao et al., “FastMRI+, Clinical pathology annotations for knee and brain fully sampled magnetic resonance imaging data,” Sci. Data, vol. 9, no. 1, p. 152, 2022.
[49] M. Lyu et al., “M4Raw: A multi-contrast, multi-repetition, multi-channel MRI k-space dataset for low-field MRI research,” Sci. Data, vol. 10, no. 1, p. 264, 2023.
[50] H. Zhao, O. Gallo, I. Frosio, and J. Kautz, “Loss Functions for Image Restoration With Neural Networks,” IEEE Trans. Comput. Imaging, vol. 3, no. 1, pp. 47-57, 2017.
[51] Z. Wang, E. P. Simoncelli, and A. C. Bovik, “Multiscale structural similarity for image quality assessment,” in Proc. Asilomar conf Signals, syst., comput., 2003, pp. 1398-1402.
[52] D. P. Kingma and J. Ba, “Adam: A Method for Stochastic Optimization.” 2017. Available: https://arxiv.org/abs/1412.6980
[53] E. G. Larsson, D. Erdogmus, R. Yan, J. C. Principe, and J. R. Fitzsimmons, “SNR-optimality of sum-of-squares reconstruction for phased-array magnetic resonance imaging,” J. Magn. Reson., vol. 163, no. 1, pp. 121-123, 2003.
[54] D. Ulyanov, A. Vedaldi, and V. Lempitsky, “Instance Normalization: The Missing Ingredient for Fast Stylization.” 2017. Available: https://arxiv.org/abs/1607.08022
[55] A. L. Maas, A. Y. Hannun, A. Y. Ng, et al., “Rectifier Nonlinearities Improve Neural Network Acoustic Models,” in Proc. Int. Conf. Mach. learn., 2013, p. 3.
[56] M. P. Recht et al., “Using Deep Learning to Accelerate Knee MRI at 3 T: Results of an Interchangeability Study,” Am. J. Roentgenol., vol. 215, no. 6, pp. 1421-1429, 2020.
[57] J. Tsao, P. Boesiger, and K. P. Pruessmann, “k-t BLAST and k-t SENSE: Dynamic MRI with high frame rate exploiting spatiotemporal correlations,” Magn. Reson. Med., vol. 50, no. 5, pp. 1031-1042, 2003.
[58] R. Kang, D. Ai, G. Qu, Q. Li, X. Li, Y. Jiang, Y. Huang, H. Song, Y. Wang, and J. Yang, “Prior information constrained alternating direction method of multipliers for longitudinal compressive sensing MR imaging,” Neurocomputing, vol. 376, pp. 128-140, 2020.
[59] A. A. Samsonov, J. V. Velikina, J. O. Fleming, M. L. Schiebler, and A. S. Field, “Accelerated serial MR imaging in multiple sclerosis using baseline scan information,” in Proc. Int. Soc. Magn. Reson. Med., 2010, p. 4876.
[60] Y. Urman, Z. Shah, A. Kumar, B. P. Soares, and K. Setsompop, “Accelerating MRI with longitudinally-informed latent posterior sampling,” Magn. Reson. Med., vol. 95, no. 6, pp. 3445-3461, 2026.
[61] A. Shamaei, A. Stebner, S. L. Bosshart, J. Ospel, G. Ginde, M. Bento, and R. Souza, “Enhancing and accelerating brain MRI through deep learning reconstruction using prior subject-specific imaging,” Magn. Reson. Imaging, 2025, Art. no. 110558.
[62] T. Oved, B. Lena, C. F. Najac, S. Shen, M. S. Rosen, A. Webb, and E. Shimron, “Deep learning of personalized priors from past MRI scans enables fast, quality-enhanced point-of-care MRI with low-cost systems,” arXiv:2505.02470, 2025.
[63] T. Karras, M. Aittala, T. Aila, and S. Laine, “Elucidating the design space of diffusion-based generative models,” in Adv. Neural Inf Process. Syst., 2022, pp. 26565-26577.
[64] A. Atalik, S. Chopra, and D. K. Sodickson, “A Trust-Guided Approach to MR Image Reconstruction with Side Information,” IEEE Trans. Med. Imaging, 2025.
[65] A. Atalik, S. Chopra, and D. K. Sodickson, “Harnessing side information for highly accelerated MRI,” in Int. Conf. Med. Image Comput. Comput.-Assist. Intervention, Cham, Switzerland: Springer Nature Switzerland, 2025, pp. 244-254.

What is claimed is:

1. A method for determining information pertaining to a structure based on measured data associated with the structure, comprising:
measuring data associated with the structure;
transforming the measured data into the information using individualized side information; and
applying a trust guidance mechanism to resolve ambiguities in the transformed information so as to prevent hallucination or overwriting of the measured data when incorporating the individualized side information.

2. The method of claim 1, wherein the trust guidance mechanism comprises incorporating the individualized side information only in an ambiguous space of the transformation.

3. The method of claim 2, wherein the incorporation comprises projecting a quantity derived from the individualized side information onto an ambiguous space associated with the transformation.

4. The method of claim 3, wherein the ambiguous space comprises a vector space associated with singular values below a predetermined threshold of a forward operator of the transformation.

5. The method of claim 1, wherein the transformation is obtained by a sequence of iterations, each iteration comprising one of a data-consistency update, a trust-guidance update via projection onto the ambiguous space, or a learned refinement update.

6. The method of claim 1, wherein the determining of the information pertaining to the structure includes an image reconstruction problem comprising generation of at least one image from the measured data.

7. The method of claim 6, wherein the at least one image includes a magnetic resonance (MR) image.

8. The method of claim 6, wherein the at least one image is reconstructed from undersampled data.

9. The method of claim 6, wherein the at least one image is reconstructed from accelerated or low-quality data arising from low-cost or accessible image acquisition hardware.

10. The method of claim 1, wherein the individualized side information comprises at least one of raw measurement data, reconstructed images, non-image data including one or more of text, tabular data, and metadata, representations learned from relevant tasks, genetic data, electronic health record data, laboratory results, clinician notes, radiology reports, pathology reports, wearable sensor measurements or a plurality of multi-modal sources used simultaneously.

11. The method of claim 6, wherein the individualized side information comprises one or more images whose contrast is the same as or different from that of the at least one reconstructed image, and are derived from a scanner or imaging modality the same as or different from that used to derive measured data for the at least one reconstructed image, and which are registered to or not registered to the at least one reconstructed image.

12. The method of claim 6, wherein the transformation comprises a trained learning model with data augmentation simulating misregistration between the individualized side information and the at least one reconstructed image, so as to reduce the effects of misregistration.

13. A system for determining information pertaining to a structure based on measured data associated with the structure, comprising:
a configuration that includes at least one sensor or at least one detector, abd configured to measure data associated with the structure; and
at least one processor configured to:
transform the measured data into the information using individualized side information; and
apply a trust guidance mechanism to resolve ambiguities in the transformed information so as to prevent hallucination or overwriting of the measured data when incorporating the individualized side information.

14. The system of claim 13, wherein the trust guidance mechanism comprises incorporating the individualized side information only in an ambiguous space of the transformation.

15. The system of claim 14, wherein the incorporation comprises projecting a quantity derived from the individualized side information onto an ambiguous space associated with the transformation.

16. The system of claim 15, wherein the ambiguous space comprises a vector space associated with singular values below a predetermined threshold of a forward operator of the transformation.

17. The system of claim 13, wherein the transformation is obtained by a sequence of iterations, each iteration comprising one of a data-consistency update, a trust-guidance update via projection onto the ambiguous space, or a learned refinement update.

18. The system of claim 13, wherein the determination of the information pertaining to the structure includes an image reconstruction problem comprising generating at least one image from the measured data.

19. The system of claim 18, wherein the at least one image includes a magnetic resonance (MR) image.

20. The system of claim 18, wherein the at least one image is reconstructed from undersampled data.

21. The system of claim 18, wherein the at least one image is reconstructed from accelerated or low-quality data arising from low-cost or accessible image acquisition hardware.

22. The system of claim 13, wherein the individualized side information comprises at least one of raw measurement data, reconstructed images, non-image data including one or more of text, tabular data, and metadata, representations learned from relevant tasks, genetic data, electronic health record data, laboratory results, clinician notes, radiology reports, pathology reports, wearable sensor measurements or a plurality of multi-modal sources used simultaneously.

23. The system of claim 18, wherein the individualized side information comprises one or more images whose contrast is the same as or different from that of the at least one reconstructed image, and are derived from a scanner or imaging modality the same as or different from that used to derive measured data for the at least one reconstructed image, and which are registered to or not registered to the at least one reconstructed image.

24. The system of claim 18, wherein the transformation comprises a trained learning model with data augmentation simulating misregistration between the individualized side information and the at least one reconstructed image, so as to reduce the effects of misregistration.

25. A non-transitory computer-accessible medium having stored thereon computer-executable instructions for determining information pertaining to a structure based on measured data associated with the structure, which when executed by a computer arrangement, configure the computer arrangement to perform procedures comprising:
measuring data associated with the structure;
transforming the measured data into the information using individualized side information; and
applying a trust guidance mechanism to resolve ambiguities in the transformed information so as to prevent hallucination or overwriting of the measured data when incorporating the individualized side information.
